(19) United States
(12) Patent Application Publication
(10) Pub. No.: US 2024/0116920 A1
(43) Pub. Date: Apr. 11, 2024

(54) NOVEL JAK-SPECIFIC INHIBITOR COMPOUND AND METHOD FOR PREPARING SAME

(71) Applicants: KOREA PHARMA CO., LTD., Hwaseong-si, Gyeonggi-do (KR); EWHA UNIVERSITY-INDUSTRY COLLABORATION FOUNDATION, Seodaemun-gu Seoul (KR)

(72) Inventors: Soo Sung KANG, Incheon (KR); Eun Sun PARK, Seoul (KR); Eun Hee PARK, Seoul (KR); Sun Joo LEE, Daegu (KR); Seung Hee HAN, Suwon-si, Gyeonggi-do (KR)

(21) Appl. No.: 18/254,927

(22) PCT Filed: Nov. 29, 2021
(86) PCT No.: PCT/KR2021/017797

§ 371 Date: May 30, 2023

(30) Foreign Application Priority Data
Nov. 30, 2020 (KR) 10-2020-0165017

(51) Int. Cl. C07D 471/04; A61P 1/16

(52) U.S. Cl. CPC C07D 471/04; A61P 1/16

(57) ABSTRACT

Provided is a novel JAK-specific inhibitor compound and a method for preparing the same. The compound of the provided can exhibit therapeutic effects on a variety of diseases, for example, inflammatory diseases, autoimmune diseases, myeloproliferative diseases, and human cancers due to its ability to regulate signal transduction at the level of JAK kinases. In particular, due to its high selectivity for JAK1, the compound of the provided can exhibit better therapeutic effects on inflammatory diseases and autoimmune diseases at a low dose and with fewer side effects and can be expected to be effective in preventing and treating liver fibrosis.

### TECHNICAL FIELD

The present invention relates to a novel JAK-specific inhibitor compound and a method for preparing the same.

### BACKGROUND ART

Protein kinases (PKs) are a group of enzymes that regulate diverse, important biological processes, including cell growth, survival and differentiation, organ formation and morphogenesis, neovascularization, tissue repair and regeneration, among others. Protein kinases exert their physiological functions through catalyzing the phosphorylation of proteins (or substrates) and thereby modulating the cellular activities of the substrates in various biological contexts. In addition to the functions in normal tissues/organs, many protein kinases also play more specialized roles in a host of human diseases, including cancer. A subset of protein kinases (also referred to as oncogenic protein kinases), when dysregulated, can cause tumor formation and growth, and further contribute to tumor maintenance and progression. Thus far, oncogenic protein kinases represent one of the largest and most attractive groups of protein targets for cancer intervention and drug development.

The Janus kinase (JAK) family plays a critical role in the cytokine-dependent regulation of proliferation and function of cells involved in immune response. At present, there are four known mammalian JAK family members: JAK1 (Janus kinase-1), JAK-2 (Janus kinase-2), JAK3 (Janus kinase-3) and TYK2 (protein-tyrosine kinase 2). The JAK proteins have a size ranging from 120 kDa to 140 kDa and comprise 7 conserved JAK homology (JH) domains. One of them is a functional catalytic kinase domain, and another is a pseudokinase domain which potentially exerts a regulatory function and/or acts as a docking site for STATs.

JAK1 is involved in most signal transduction pathways mediated by cytokines such as γc cytokines (IL-2, IL-4, IL-7, IL-9, IL-21), members of the gp130 cytokine family (IL-6, IL-11, LIF, OSM), members of the IRF family, and IL-10-like cytokines and regulates the functions of T-cells and B-cells (Non-Patent Document 1). Thus, JAK1 is being developed for the treatment of diseases such as rheumatoid arthritis, inflammatory bowel disease, atopic dermatitis, and psoriasis.

JAK2 is used for signal transduction in which cytokines such as Pc cytokines, gp130 cytokine family members, EPO, IRF, IL-12, and IL-23 are involved. Particularly, when cytokines such as EPO, TPO, IL-3, and IL-5 bind to their receptors, JAK2 kinases tend to homodimerize and are ultimately involved in myeloid differentiation and haematopoiesis as well as T cell proliferation. Thus, selective JAK2 inhibitors are being developed for the treatment of myeloproliferative disorders such as myeloproliferative neoplasms (Non-Patent Document 2).

The expression of JAK3 is more restricted than that of other JAK kinases. When yc cytokines bind to their receptors, JAK3 is involved only in a single pathway through dimerization with JAK1. Thus, JAK3 inhibitors can be used to treat diseases such as rheumatoid arthritis and psoriasis (Non-Patent Document 3). JAK3 selective inhibitors are likely to cause fewer side effects than other inhibitors, but their efficacy may also be low compared to other inhibitors because they are involved only in a single pathway among various immune-related JAK/STAT pathways (Non-Patent Document 4).

Furthermore, blocking signal transduction at the level of JAK kinases also hold promise for developing treatments for various diseases, for example, inflammatory diseases, autoimmune diseases, myeloproliferative diseases, and human cancers and may result in therapeutic benefits in patients suffering from immune disorders of the skin such as psoriasis and skin sensitization.

Accordingly, inhibitors of Janus kinases or related kinases are widely sought and several patent publications report effective classes of compounds. For example, certain JAK inhibitors, including pyrrolopyridine and pyrrolopyrimidine compounds, are disclosed in U.S. Patent Publication No. 2007/0135461 A1 (Patent Document 1).

The entire disclosure of all prior art documents cited above is incorporated herein by reference.

### PRIOR ART DOCUMENTS

### Patent Documents

(Patent Document 1) US 2007/0135461 A1 (Jun. 14, 2007)

### Non-Patent Documents

(Non-Patent Document 1) SCHWARTZ, Daniella M., et al. Type I/II cytokines, JAKs, and new strategies for treating autoimmune diseases. Nature Reviews Rheumatology, 2016, 12.1: 25.

(Non-Patent Document 2) PURANDARE, A. V., et al. Characterization of BMS-911543, a functionally selective small-molecule inhibitor of JAK2. Leukemia, 2012, 26.2: 280.

(Non-Patent Document 3) VAINCHENKER, William, et al. JAK inhibitors for the treatment of myeloproliferative neoplasms and other disorders. F1000Research, 2018, 7.

(Non-Patent Document 4) THOMA, Gebhard; DRUECKES, Peter; ZERWES, Hans-Guenter. Selective inhibitors of the Janus kinase Jak3—Are they effective?, Bioorganic & medicinal chemistry letters, 2014, 24.19: 4617-4621.

### DETAILED DESCRIPTION OF THE INVENTION

### Problems to be Solved by the Invention

As described above, JAK kinases such as JAK1, JAK2, and JAK3 have their own characteristics. The present inventors have intended to develop a drug that selectively inhibits JAK1 so as to be more effective against most inflammation and autoimmune related diseases for better efficacy.

However, it is difficult to enhance the activities of ATP-binding kinase domains of all JAK family members for specific kinases because the structural differences of the kinase domains are not very significant.

The present inventors have referred to previously developed inhibitors whose structures are known to choose an initial structure, and as a result, succeeded in finding an inhibitor that not only exhibits high affinity for JAK1, but also acts more selectively on JAK1 than on other JAK kinases. The present invention has been accomplished based on this finding. An object of the present invention is to provide a novel compound that has high inhibitory activity against Janus kinases, particularly JAK1.

### Means for Solving the Problems

The present invention has been made in an effort to solve the problems of the prior art.

The present invention provides a compound represented by Formula I:

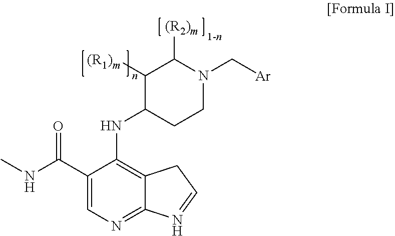

wherein

R₁ is C₁-C₃ alkyl or halogen,

R₂ is C₁-C₃ alkyl,

Ar is phenyl or heteroaryl unsubstituted or substituted with one or more substituents selected from the group consisting of C₁-C₃ alkyl, C₁-C₃ alkoxy, halogen, cyan, and —CF₃,

m is 1 or 2, and n is 0 or 1, or a pharmaceutically acceptable salt thereof.

The heteroaryl may be pyridine or pyrimidine.

The compound of Formula I may be represented by Formula II:

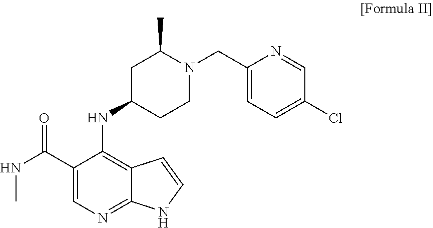 [SMILES: CNC(=O)c1cnc2[nH]ccc2c1N[C@@H]1CCN(Cc2ccc(Cl)cn2)[C@H](C)C1]

The compound of Formula I may be represented by Formula III:

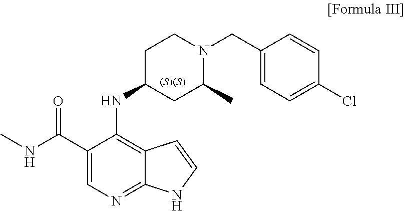

The compound of Formula I may be represented by Formula IV:

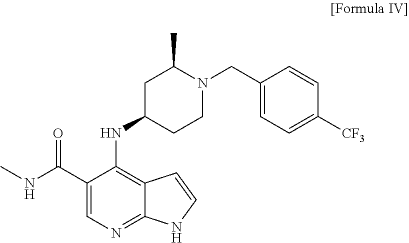 [SMILES: CNC(=O)c1cnc2[nH]ccc2c1N[C@@H]1CCN(Cc2ccc(C(F)(F)F)cc2)[C@H](C)C1]

The compound of Formula I may be represented by Formula V:

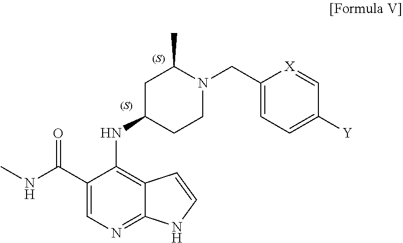

wherein X is CH or nitrogen and Y is C₁-C₃ alkyl, C₁-C₃ alkoxy, halogen, cyan or —CF₃.

The present invention also provides a pharmaceutical composition for treating or preventing an autoimmune disease or cancer including the compound represented by Formula I or pharmaceutically acceptable salt thereof.

The present invention also provides a pharmaceutical composition for treating or preventing liver fibrosis including the compound represented by Formula I or pharmaceutically acceptable salt thereof.

### Effects of the Invention

The compound of the present invention can exhibit therapeutic effects on a variety of diseases, for example, inflammatory diseases, autoimmune diseases, myeloproliferative diseases, and human cancers due to its ability to regulate signal transduction at the level of JAK kinases. In particular, due to its high selectivity for JAK1, the compound of the present invention can exhibit better therapeutic effects on inflammatory diseases and autoimmune diseases at a low dose and with fewer side effects and can be expected to be effective in preventing and treating liver fibrosis.

### BRIEF DESCRIPTION OF THE DRAWINGS

FIG. 1a shows proliferation of LX-2 induced by TGF-β, which was examined using CCK8.

FIG. 1b shows the GI₅₀ values of nintedanib and an inventive compound 31g, which were measured under the same conditions.

FIG. 1c shows the inhibitions of LX-2 cell growth evaluated after incubation of nintedanib or 31g with TGF-β for 72 h.

FIGS. 2a and 2b show the results of cytotoxicity evaluation using cell impermeable YOYO1.

FIG. 3a shows the gene expression levels of fibrotic parameters measured after incubation of LX-2 with untreated TGF-β or TGF-β treated with an inventive compound 31g (500 nM).

FIG. 3b shows the results of immunofluorescence analysis of α-SMA (green) and Hoechest 33258 (blue) in LX-2 cells treated with 10 ng/ml TGF-β for 72 h (scale bar=200 μm, right: fluorescence intensities analyzed as phenotypic percentages of a population with a constant GFP intensity using Harmony 3.1).

FIG. 3c shows the inhibitory effect of an inventive compound (31g) on wound healing/migration, which was evaluated based on an in vitro scratch wound assay using LX-2 cells (scratch wound healing/migration induced by TGF-β in LX-2 cells treated with medium alone (0.5% FBS) was expressed as the concentration of 31g (red line: initial (0 h), purple line: after 24 h).

FIG. 4 shows the results of immunoassay for Immunobit cell-based pSTAT3 and pSTAT5 in TF1 cells treated with nintedanib and an inventive compound 31g (250 nM).

FIG. 5 shows TGF-β-induced STAT3 phosphorylation levels in LX-2 after treatment with an inventive compound 31g (250 nM); band intensities were analyzed using ODYSSEY Image Studio software (LI-COR).

### BEST MODE FOR CARRYING OUT THE INVENTION

The present invention will now be described in detail.

One aspect of the present invention is directed to a compound represented by Formula I:

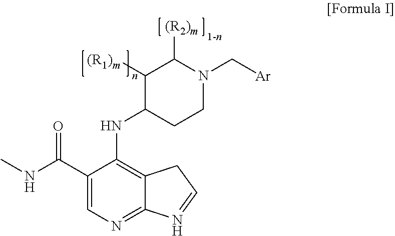

wherein

R₁ is C₁-C₃ alkyl or halogen,

R₂ is C₁-C₃ alkyl,

Ar is phenyl or heteroaryl unsubstituted or substituted with one or more substituents selected from the group consisting of C₁-C₃ alkyl, C₁-C₃ alkoxy, halogen, cyan, and —CF₃,

m is 1 or 2, and n is 0 or 1, or a pharmaceutically acceptable salt thereof.

The heteroaryl may be pyridine or pyrimidine.

More specifically, the compound of by Formula I may be represented by Formulae II, III, and IV:

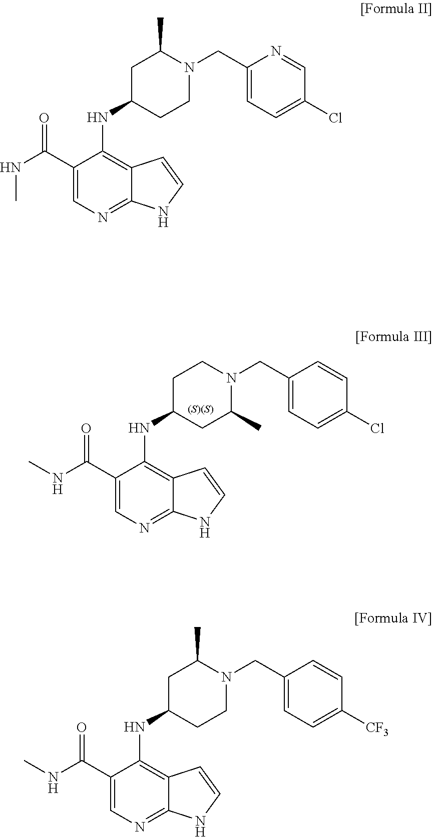

The compound of the present invention may have one or more asymmetric centers. Unless otherwise indicated, all chiral (enantiomeric and diastereomeric) and racemic forms of the compound according to the present invention are within the scope of the present invention.

Many geometric isomers such as olefins and C═N double bonds may also exist in the compound and all stable isomers can be contemplated in the present invention. Cis and trans geometric isomers of the compound according to the present invention are described and may be isolated as a mixture of isomers or as separated isomeric forms.

The compound of the present invention may be isolated in optically active or racemic forms. Methods for preparing optically active forms (for example, by resolution of racemic forms or by synthesis from optically active starting materials) are widely known in the art.

All chiral (enantiomeric and diastereomeric), racemic forms and all geometric isomeric forms of a structure are intended, unless the specific stereochemistry or isomeric form is specifically indicated.

A specific isomeric form of the compound according to the present invention may be represented by Formula V:

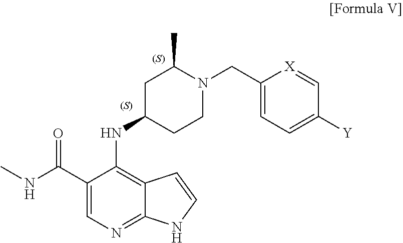

wherein X is CH or nitrogen and Y is C₁-C₃ alkyl, C₁-C₃ alkoxy, halogen, cyan or —CF₃, or a pharmaceutically acceptable salt thereof.

The compound represented by Formula I can regulate the activity of Janus kinases (JAKs). As used herein, the term “regulate” refers to the ability to increase or decrease the activity of one or more kinases of the JAK family. Due to this ability, the compound of the present invention or a composition including the compound can be brought into contact with JAKs to regulate the JAKs. In some particular embodiments, the compound of the present invention may act as an inhibitor of one or more JAKs.

The compound of the present invention binds to and/or regulates JAKs, including any members of the JAK family. In some embodiments, the JAK is JAK1, JAK2, JAK3 or TYK2. In some embodiments, the JAK is JAK1 or JAK2. In some embodiments, the JAK is JAK1. In some embodiments, the JAK is JAK1 or JAK3.

Another aspect of the present invention is directed to a pharmaceutical composition for

treating or preventing an autoimmune disease, an inflammatory disease or a cancer including the compound represented by Formula I or pharmaceutically acceptable salt thereof.

JAK-associated diseases include diseases, disorders, and conditions that are directly or indirectly associated with JAK expression or activity (for example, overexpression and/or aberrant activity). JAK-associated diseases may be, for example, diseases, disorders, and conditions that can be prevented, ameliorated or treated by regulation of JAK activity.

Examples of JAK-associated diseases include immune system-related diseases such as rejection reactions after organ transplantation (e.g., transplant rejection and graft versus host responses), multiple sclerosis, rheumatoid arthritis, juvenile arthritis, psoriatic arthritis, type I diabetes, lupus, psoriasis, inflammatory bowel disease, ulcerative colitis, Crohn's disease, severe myasthenia gravis, immunoglobulin nephropathy, autoimmune thyroid disorders, asthma, food allergy, atopic dermatitis, psoriasis, and autoimmune bullous skin disorders such as pemphigus vulgaris (PV) and bullous pemphigoid (BP).

Other examples of JAK-associated diseases include inflammation and inflammatory diseases. Examples of inflammatory diseases include inflammatory diseases of the eye (e.g., iritis, uveitis, scleritis, conjunctivitis or related diseases), inflammatory diseases of the respiratory tract (e.g., rhinitis or sinusitis of the upper respiratory tract including the nose and sinuses and bronchitis and chronic obstructive pulmonary diseases of the lower respiratory tract), inflammatory myopathy such as myocarditis, and other inflammatory diseases. The pharmaceutical composition can also be used to treat or prevent other inflammatory diseases, for example, systemic inflammatory response syndrome (SIRS) and septic shock.

Other examples of JAK-associated diseases include cancers characterized by solid tumors (e.g., prostate cancer, renal cancer, hepatic cancer, pancreatic cancer, gastric cancer, breast cancer, lung cancer, head and neck cancer, thyroid cancer, glioblastoma, Kaposi's sarcoma, Castleman's disease, and melanoma), hematologic cancers (e.g., lymphoma, leukemia such as acute lymphocytic leukemia, acute myeloid leukemia (AML), and multiple myeloma), and skin cancers such as cutaneous T-cell lymphoma (CTCL) and cutaneous B-cell lymphoma. Examples of cutaneous T-cell lymphomas include Sezary syndrome and mycosis fungoides.

The JAK inhibitor of the present invention can also be used to treat gout and increased prostate size, for example, due to benign prostatic hypertrophy or prostatic hyperplasia. The JAK inhibitor of the present invention can also be used to treat a disease or condition associated with ischemic reperfusion injury or an inflammatory ischemic event such as stroke or cardiac arrest. The JAK inhibitor of the present invention can also be used to treat stenosis and sclerodermatitis.

The JAK inhibitor of the present invention can also be used to treat a condition associated with hypoxia or astrogliosis, for example, diabetic retinopathy, cancer or neurodegeneration. See Dudley, A.C. et al. Biochem. J. 2005, 390(Pt 2):427-36 and Sriram, K. et al. J. Biol. Chem. 2004, 279(19): 19936-47. Epub 2004 Mar. 2. The JAK inhibitor of the present invention can also be used to treat Alzheimer's disease.

Furthermore, the JAK inhibitor of the present invention can be used to prevent or treat fibrosis. In particular, the JAK inhibitor of the present invention was confirmed to have the potential to prevent or treat liver fibrosis. A more detailed understanding can be obtained from the Examples section that follows.

The pharmaceutical composition of the present invention may be formulated into tablets, capsules (including sustained release or timed release preparations), pills, powders, granules, elixirs, tinctures, suspensions, syrups, and emulsions for oral administration. These preparations may also be administered intravenously (by bolus injection or infusion), intraperitoneally, subcutaneously or intramuscularly. All available dosage forms are well known to those skilled in the pharmaceutical art. The dosage forms can be administered alone but will generally be administered with a pharmaceutical carrier selected based on the chosen route of administration and standard pharmaceutical practice.

The pharmaceutical composition of the present invention may be administered intranasally through topical use of a suitable intranasal vehicle or via a transdermal route using a transdermal skin patch. To be administered in the form of a transdermal delivery system, the dosage administration will, of course, be continuous rather than intermittent throughout the dosage regimen.

### EXAMPLES

### Confirmation of Effects as JAK Inhibitors

Compounds that can be represented by Formula I were synthesized. Some of the compounds are shown in Tables 1-6. The IC₅₀ values of the compounds for JAK kinases were measured and are shown in Tables 1-6. However, the specific examples of the compounds are given to provide complete disclosure of the invention and aid those skilled in the art in understanding the invention and are not intended to limit the scope of the invention.

TABLE 1
43a-f

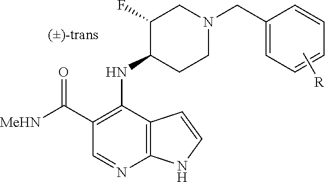

44a-f

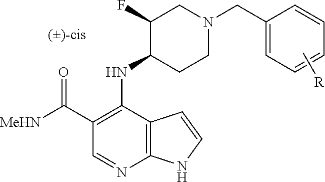

| | | IC₅₀ (nM) | | | |
|---|---|---|---|---|---|
| | R | JAK1 | JAK2 | JAK3 | TYK2 |
| tofacitinib | — | 1.26 | 9.63 | 0.99 | 12.4 |
| filgotinib | — | 16.2 | 206.3 | 321.8 | 71.5 |
| 43a | H | 35.33 | — | — | — |
| 43b | 4-Cl | 48.42 | — | — | — |
| 43c | 3-OMe | 16.49 | 203.34 | 305.2 | 493.2 |
| 43d | 4-OMe | 73.62 | | | |
| 43e | 3-CN | 14.97 | 180.7 | 143.4 | 1070 |
| 43f | 4-CN | 11.80 | 255.9 | 316.1 | 411.9 |
| 44a | H | 11.99 | | | |
| 44b | 3-Cl | 6.39 | 88.1 | 249.6 | 593.1 |
| 44c | 4-Cl | 4.24 | 87.8 | 115.5 | 458.5 |
| 44d | 4-OMe | 7.27 | 187.8 | 363.6 | 1124.9 |
| 44e | 3-CN | 3.15 | 64.9 | 250.9 | 357.6 |
| 44f | 4-CN | 8.32 | 88.0 | 221.1 | 239.2 |

TABLE 2
45a-g

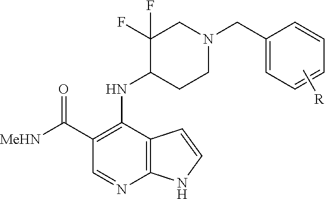

| | | IC₅₀ (nM) | | | |
|---|---|---|---|---|---|
| | R | JAK1 | JAK2 | JAK3 | TYK2 |
| 45a | H | 105.05 | | | |
| 45b | 3-Cl | 63.18 | | | |
| 45c | 4-Cl | 45.54 | 761.8 | 664.4 | 1069.5 |
| 45d | 3-OMe | 365.01 | | | |
| 45e | 4-OMe | 117.59 | | | |
| 45f | 3-CN | 205.39 | | | |
| 45g | 4-CN | 251.98 | | | |

TABLE 3
46a-d

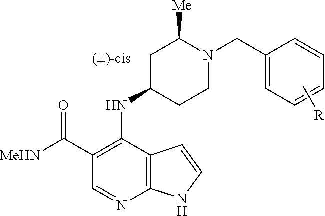

| | | IC₅₀ (nM) | | | |
|---|---|---|---|---|---|
| | R | JAK1 | JAK2 | JAK3 | TYK2 |
| 46a | 2-F | 12.97 | 18.7 | 12.0 | 38.7 |
| 46b | 3-F | 21.21 | 32.9 | 17.6 | 75.9 |
| 46c | 4-F | 3.44 | 8.97 | 10.8 | 8.42 |
| 46d | 4-CF₃ | 0.252 | 43.2 | 14.5 | 123.4 |

TABLE 4
47a-d

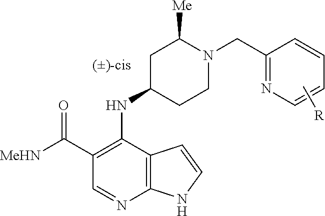

48a-c

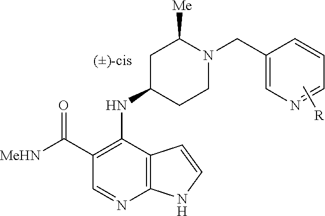

| | | IC₅₀ (nM) | | | |
|---|---|---|---|---|---|
| | R | JAK1 | JAK2 | JAK3 | TYK2 |
| 47a | 5-Cl | 0.734 | 7.39 | 3.63 | 40.3 |
| 47b | 6-CN | 3.75 | 22.6 | 22.7 | 16.6 |
| 47c | 5-CN | 6.722 | 24.5 | 11.6 | 44.9 |
| 47d | 5-CF₃ | 1.943 | 15.8 | 6.93 | 57.2 |
| 48a | 6-Cl | 3.31 | 13.1 | 9.11 | 31.6 |
| 48b | 6-CN | 1.474 | 8.50 | 4.81 | 13.1 |
| 48c | 6-CF₃ | 3.649 | 27.1 | 40.5 | 74.9 |

TABLE 5
49

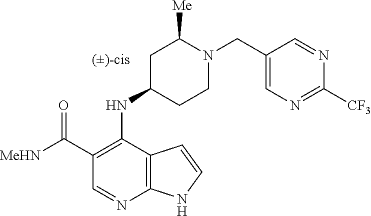 [SMILES: CNC(=O)c1cnc2[nH]ccc2c1N[C@@H]1CCN(Cc2cnc(C(F)(F)F)nc2)[C@H](C)C1]

50a-c

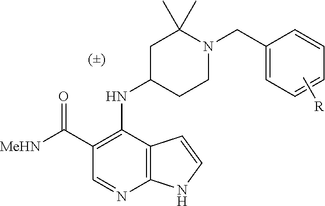

| | | IC₅₀ (nM) | | | |
|---|---|---|---|---|---|
| | R | JAK1 | JAK2 | JAK3 | TYK2 |
| 49 | 2-CF₃ | 3.04 | 8.78 | 8.39 | 36.9 |
| 50a | H | 54.67 | | | |
| 50b | 3-Cl | 17.07 | 99.6 | 166.8 | 395.3 |
| 50c | 4-Cl | 124.57 | | | |

TABLE 6

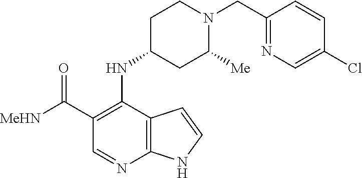 [SMILES: CNC(=O)c1cnc2[nH]ccc2c1N[C@@H]1CCN(Cc2ccc(Cl)cn2)[C@H](C)C1]

47a (±)-cis mixture
56a (S,S)-enantiomer
56b (R,R)-enantiomer

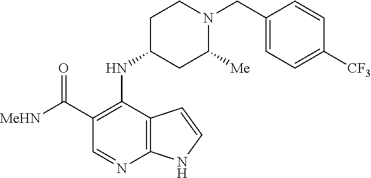 [SMILES: CNC(=O)c1cnc2[nH]ccc2c1N[C@@H]1CCN(Cc2ccc(C(F)(F)F)cc2)[C@H](C)C1]

46d (±)-cis-mixture
57a (S,S)-enantiomer
57b (R,R)-enantiomer

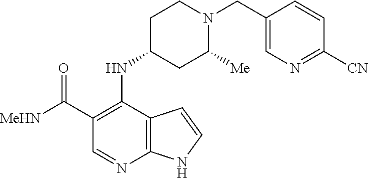 [SMILES: CNC(=O)c1cnc2[nH]ccc2c1N[C@@H]1CCN(Cc2ccc(C#N)nc2)[C@H](C)C1]

48b (±)-cis mixture
56a (S,S)-enantiomer
58b (R,R)-enantiomer

| | IC₅₀ (nM) | | | |
|---|---|---|---|---|
| | JAK1 | JAK2 | JAK3 | TYK2 |
| 56a | 0.552 | 6.68 | 8.05 | 9.32 |
| 56b | 181.38 | | | |
| 57a | 0.017 | 16.8 | 9.24 | 58.3 |
| 57b | 112.4 | 865.5 | 957.4 | 788.6 |
| 58a | 0.577 | | | |
| 58b | 56.1 | | | |

TABLE 7

GI₅₀ (μM)

| | JAK1 | JAK2 | JAK3 | TYK2 |
|---|---|---|---|---|
| tofacitinib | 0.0440 | 1.382 | 0.462 | 0.302 |
| 56a | 0.0288 | 0.575 | 1.80 | 0.0994 |
| 57a | 0.00145 | 0.577 | 1.41 | 0.174 |
| 58a | 0.0192 | 0.934 | 2.08 | 0.164 |

### Ba/F3 Cell-based Assays of the Inhibitors

The compounds that can be represented by Formula I, including the compounds exemplified above, can be prepared by various methods, including methods specifically described in the general synthetic procedures that follow. Those skilled in the art can estimate and understand synthetic methods for specific exemplary compounds and the compounds described in the Examples section based on the description of the general synthetic procedures, and thus explanations of individual methods for preparing the compounds are omitted.

### General Synthetic Procedures

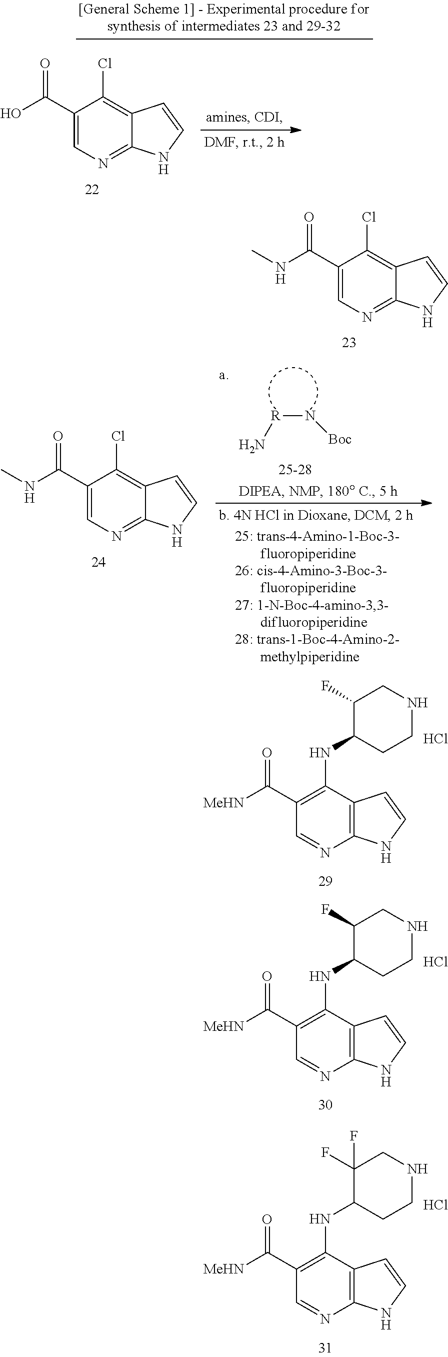

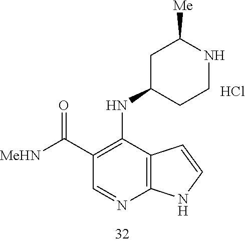 [SMILES: CNC(=O)c1cnc2[nH]ccc2c1N[C@@H]1CCN[C@H](C)C1 ; Cl]

As depicted in General Scheme I, N-methylated 1-H-pyrrolo[2,3-b]pyridine carboxamides were synthesized by carbonyldiimidazole-mediated amide coupling of 4-chloro-1H-pyrrolo[2,3-b]pyridine-5-carboxylic acid. Compounds 29-32 were prepared by microwave-assisted nucleophilic substitution of chlorine at the C4-position of the compound 23 with various amines 25-28. The amines were Boc-deprotected with hydrochloric acid to prepare intermediates 29-32 for the preparation of final products 43-49.

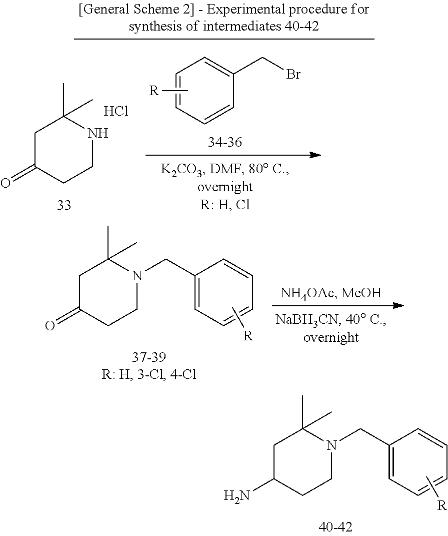

As depicted in General Scheme 2, the 2-dimethyl piperidine intermediates were prepared by N-benzylation of starting material 33 with K₂CO₃ as a base and subsequent reductive amination with NH₄OAc.

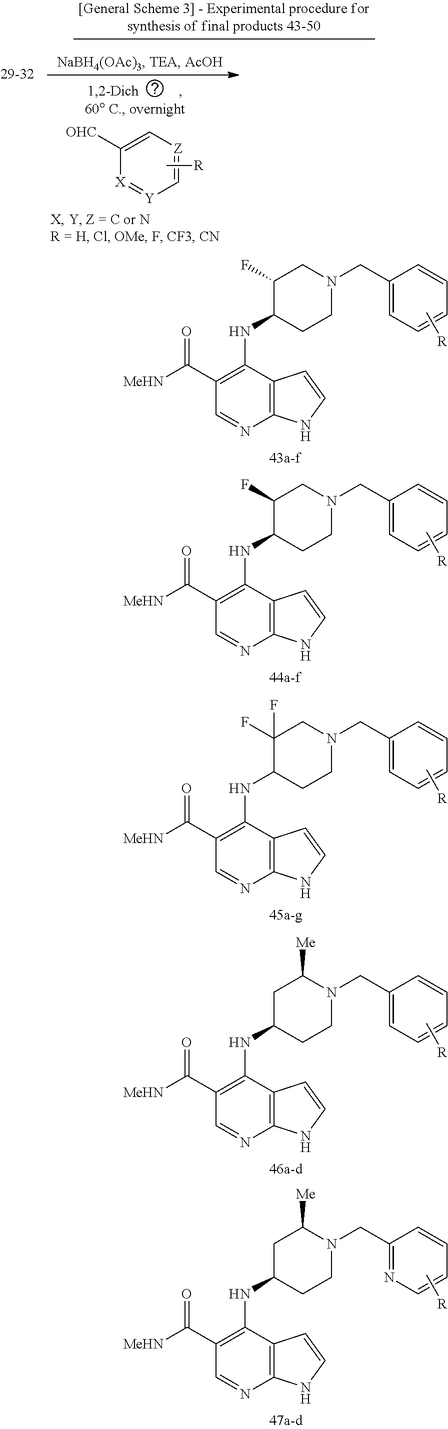

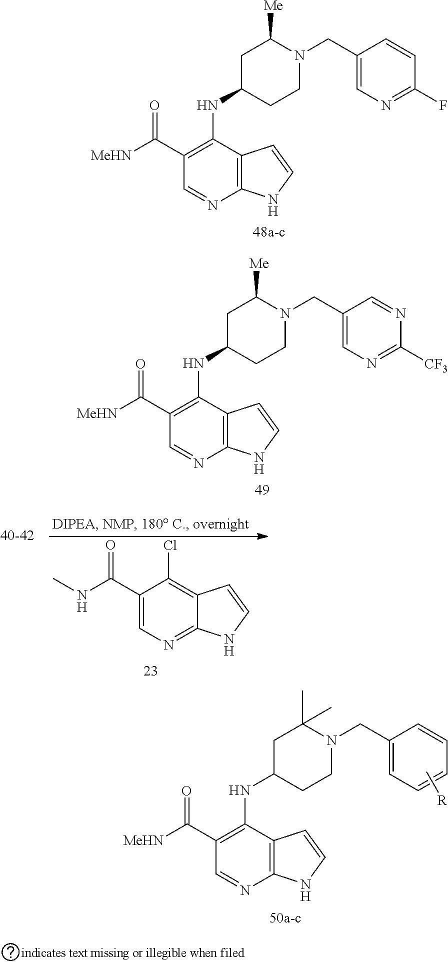

As depicted in General Scheme 3, the Boc-deprotected intermediates 29-32 were subjected to reductive amination with various aldehydes to synthesize the final products 43-49. Nucleophilic substitution with the amine intermediates 40-42 under microwave conditions afforded the final products 50.

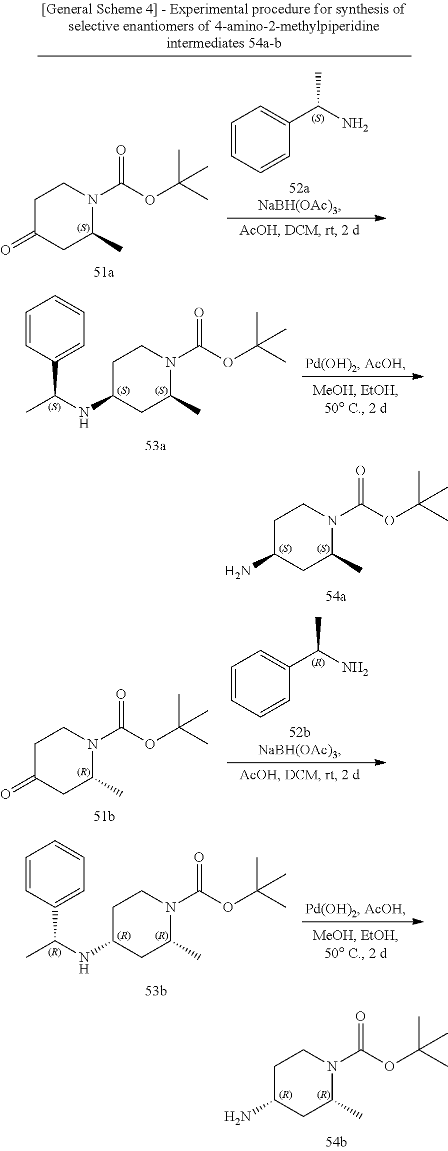

As depicted in General Scheme 4, 51a and 51b and enantiomers 52a and 52b were used to prepare the enantiomeric intermediates 53a and 53b, respectively, for selective control over the chiralities at positions 2 and 4 of 4-aminopiperidine. The selective enantiomers 53a and 53b were subjected to debenzylation by reduction under palladium conditions to afford the intermediates 54a and 54b, respectively.

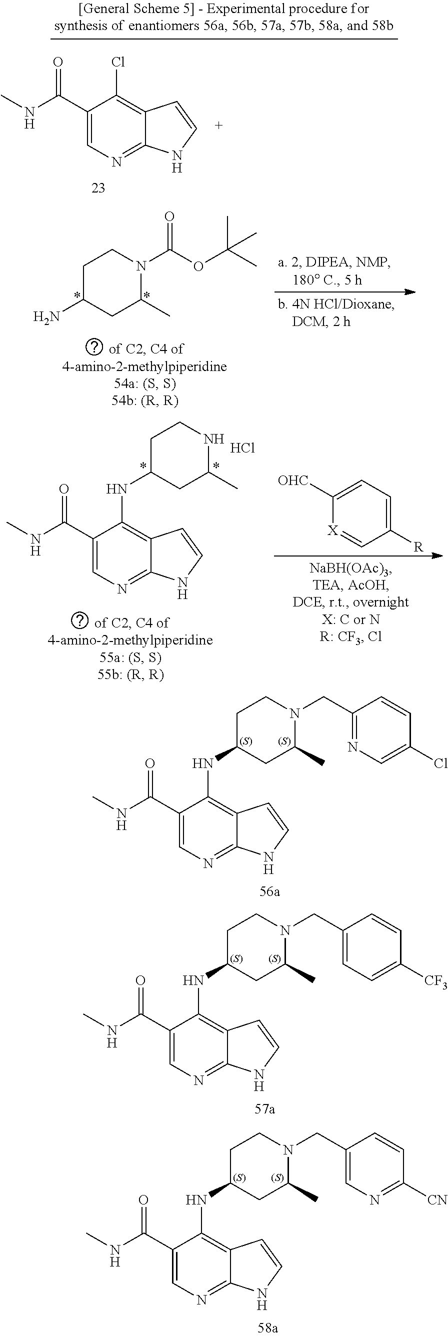

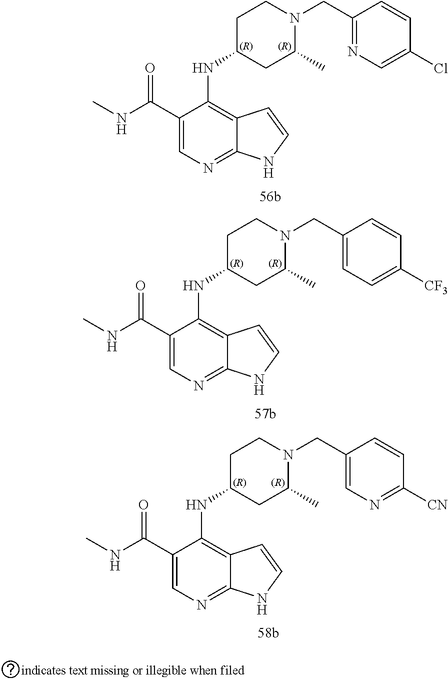

As depicted in General Scheme 5, the intermediate 23 as a basic skeleton and the enantiomeric amines 54a and 54b were subjected to microwave-assisted nucleophilic substitution and reductive amination in the same manner as in General Schemes 1 and 5 to give the final products 56a, 56b, 57a, 57b, 58a, and 58b.

### Synthesis of 4-chloro-N-methyl-1H-pyrrolo[2,3-b]pyridine-5-carboxamide (23)

4-Chloro-1H-pyrrolo[2,3-b]pyridine-5-carboxylic acid (1 eq) and CDI (1.05 eq) were added to 20 ml of DMF under a nitrogen atmosphere at room temperature. The mixture was stirred at the same temperature for 1 h. To the mixture was slowly added dropwise methylamine (5 eq). The resulting mixture was stirred at room temperature for 1 h. The progress of the reaction was monitored by TLC and the DMF solvent was removed using a rotary evaporator. Ethyl acetate was poured into the resulting mixture. The precipitate was collected by filtration and washed with water to remove the imidazole. Filtration afforded the title product as a white solid. ¹H NMR (400 MHz, DMSO-d₆) δ 12.15 (s, 1H), 8.37 (q, J=4.7 Hz, 1H), 8.24 (s, 1H), 7.65 (d, J=3.5 Hz, 1H), 6.56 (d, J=3.4 Hz, 1H), 2.80 (d, J=4.6 Hz, 3H); ¹³C NMR (100 MHz, chloroform-d) δ 168.85, 149.42, 143.10, 134.32, 128.48, 124.54, 120.69, 100.59, 26.93; MS (ESI, m/z) calculated for C₉H₉ClN₃O [M+H]⁺ 209.04, found 210.00.

### General Procedure A

DIEA (2 eq) and chloro-N-methyl-1H-pyrrolo[2,3-b]pyridine-5-carboxamide (1.0 mmol) were added to the Boc-protected amines 25-28 and 54a-b (2 eq) in a solvent (NMP, 2 mL). The mixture was stirred under microwave irradiation at 180° C. The resulting mixture was cooled to room temperature, diluted with ethyl acetate, washed with H₂O and brine, and dried over MgSO₄. The crude mixture was purified by silica gel flash chromatography (dichloromethane:MeOH=10:1 elution) to afford the title products. 4 N HCl in dioxane (2 mL) was added to a solution of the compounds 25-28 and 54a-b in MeOH. The mixture was stirred at room temperature for 2 h for Boc deprotection. The products were used in the next step without further purification.

### 4-((trans-3-Fluoropiperidin-4-yl)amino)-N-methyl-1H-pyrrolo[2,3-b]pyridine-5-carboxamide hydrochloride (29)

The title compound was synthesized according to General Procedure A. Yield: 26.1%; ¹H NMR (400 MHz, Methanol-d₄) δ 8.46 (s, 1H), 7.40 (d, J=3.7 Hz, 1H), 7.01 (d, J=3.7 Hz, 1H), 5.17-5.06 (m, 1H), 5.04-4.94 (m, 1H), 3.63-3.57 (m, 2H), 2.93 (s, 3H), 2.61-2.49 (m, 2H), 2.17-2.06 (m, 2H); ¹³C NMR (100 MHz, Methanol-d₄) δ 169.32, 154.26, 139.88, 135.89, 125.50, 108.37, 106.48, 105.19, 87.31 (d, J_{C-F}=181.0 Hz), 51.81 (d, J_{C-F}=23.8 Hz), 44.84 (d, J_{C-F}=26.3 Hz), 41.59, 26.80, 26.24; MS (ESI, m/z) calculated for C₁₄H₁₉FN₅O [M+H]⁺ 292.16, found 292.10.

### 4-((cis-3-Fluoropiperidin-4-yl)amino)-N-methyl-1H-pyrrolo[2,3-b]pyridine-5-carboxamide hydrochloride (30)

The title compound was synthesized according to General Procedure A. Yield: 78.8%; ¹H NMR (400 MHz, DMSO-d₆) δ 11.52 (s, 1H), 9.59 (d, J=8.7 Hz, 1H), 8.31 (s, 1H), 8.23 (d, J=4.4 Hz, 1H), 7.16 (d, J=3.5 Hz, 1H), 6.53 (d, J=3.6 Hz, 1H), 4.71 (d, J=50.5 Hz, 1H), 4.34-4.14 (m, 1H), 3.12 (t, J=11.4 Hz, 1H), 2.90 (d, J=14.9 Hz, 1H), 2.84-2.76 (m, 1H), 2.74 (d, J=4.4 Hz, 3H), 2.65 (t, J=12.0 Hz, 1H), 1.86-1.75 (m, 1H), 1.67-1.51 (m, 2H); ¹³C NMR (100 MHz, DMSO-d₆) δ 170.02, 150.34, 148.53, 144.51, 122.04, 104.62, 103.13, 101.54, 88.88 (d, J_{C-F}=173.6 Hz), 51.71 (d, J_{C-F}=18.1 Hz), 48.23 (d, J_{C-F}=20.5 Hz), 29.62, 28.05, 26.09; MS (ESI, m/z) calculated for C₁₄H₁₉FN₅O [M+H]⁺ 292.16, found 292.10.

### 4-((3,3-Difluoropiperidin-4-yl)amino)-N-methyl-1H-pyrrolo[2,3-b]pyridine-5-carboxamide hydrochloride (31)

The title compound was synthesized according to General Procedure A. Yield: 89.1%; ¹H NMR (400 MHz, chloroform-d) δ 12.09 (s, 1H), 8.93 (s, 1H), 8.54 (d, J=4.7 Hz, 1H), 7.38 (d, J=3.6 Hz, 1H), 6.63 (d, J=3.6 Hz, 1H), 3.92-3.82 (m, 1H), 3.59-3.51 (m, 1H), 3.49 (s, 3H), 3.31 (t, J=11.0 Hz, 1H), 3.27-3.16 (m, 1H), 3.03 (d, J=4.8 Hz, 3H), 2.20-2.10 (m, 1H), 1.78-1.69 (m, 1H), 1.46 (d, J=6.5 Hz, 1H); ¹³C NMR (100 MHz, chloroform-d) δ 167.37, 151.57, 149.10, 146.45, 125.61, 122.48 (t, J_{C-F}=244.4 Hz), 116.47, 114.41, 100.57, 55.10 (dd, J_{C-F}=31.5, 26.4 Hz), 52.38 (t, J_{C-F}=22.8 Hz), 51.17, 31.20, 26.33; MS (ESI, m/z) calculated for C₁₄H₁₈F₂N₅O [M+H]⁺ 310.15, found 310.15.

### N-Methyl-4-((cis-2-methylpiperidin-4-yl)amino)-1H-pyrrolo[2,3-b]pyridine-5-carboxamide hydrochloride (32)

The title compound was synthesized according to General Procedure A. Yield: 37.6%; ¹H NMR (400 MHz, Deuterium Oxide) δ 8.10 (d, J=0.9 Hz, 1H), 7.19 (dd, J=3.7, 0.8 Hz, 1H), 6.51 (dd, J=3.8, 0.9 Hz, 1H), 4.43-4.37 (m, 1H), 3.51-3.41 (m, 1H), 3.42-3.35 (m, 1H), 3.23-3.11 (m, 1H), 2.90 (d, J=0.8 Hz, 3H), 2.15-2.00 (m, 2H), 1.99-1.88 (m, 1H), 1.48-1.43 (m, 1H), 1.35 (d, J=6.5 Hz, 3H); ¹³C NMR (100 MHz, Deuterium oxide) δ 171.4, 148.6, 148.5, 143.5, 122.4, 104.9, 103.1, 101.7, 47.6, 44.2, 39.0, 34.6, 27.5, 26.7, 26.0; HRMS (ESI, m/z) calculated for C₁₅H₂₂N₅O [M+H]⁺ 288.1819, found 288.1821.

### General Procedure B

K₂CO₃ (5.2 mmol) and various benzyl bromides (2.6 mmol) were added to a solution of 2,2-dimethylpiperidin-4-one (1.3 mmol) in DMF (5 ml). The resulting mixture was allowed to react at 80° C. for at least 12 h. The reaction mixture was diluted with DCM (40 mL), washed with H₂O and brine, dried over MgSO4, and purified by silica gel flash chromatography (30% ethyl acetate in hexane elution) to afford the title products 37-39. For amine substitution of the carbonyl groups of the intermediates 37-39, ammonium acetate (10 eq) and 4 A molecular sieves were added to a solution of 37-39 (1 eq) in 10 mL of methanol. The mixture was stirred for 1 h. To the mixture was added sodium cyanoborohydride (2 eq). The resulting mixture was further stirred at room temperature for at least 12 h. The reaction mixture was diluted with DCM (40 ml), washed with H₂O and brine, dried over MgSO₄, and purified by silica gel flash chromatography (dichloromethane:methanol=10:1) to afford the title amine products 40-42.

### 1-Benzyl-2,2-dimethylpiperidin-4-one (37)

The title compound was synthesized according to General Procedure B. Yield: 41.4%; ¹H NMR (400 MHz, chloroform-d) δ 7.39 (d, J=7.1 Hz, 2H), 7.33 (t, J=7.4 Hz, 2H), 7.25 (t, J=7.2 Hz, 1H), 3.63 (s, 2H), 2.76 (t, J=6.3 Hz, 2H), 2.39 (s, 2H), 2.36-2.28 (m, 2H), 1.19 (s, 6H); ¹³C NMR (100 MHz), chloroform-d) δ 210.35, 140.46, 128.43, 128.40, 127.01, 57.79, 55.61, 52.92, 46.29, 41.62, 24.07; MS (ESI, m/z) calculated for C₁₄H₂₀NO [M+H]⁺ 218.15, found 218.10.

### 1-(3-Chlorobenzyl)-2,2-dimethylpiperidin-4-one (38)

The title compound was synthesized according to General Procedure B. Yield: 45.6%; ¹H NMR (400 MHz, chloroform-d) δ 7.43-7.40 (m, 1H), 7.27-7.21 (m, 3H), 3.61 (s, 2H), 2.75 (t, J=6.3 Hz, 2H), 2.39 (s, 2H), 2.34 (dd, J=6.4, 1.4 Hz, 2H), 1.18 (s, 6H); ¹³C NMR (100 MHz, 15 chloroform-d) δ 210.02, 142.80, 134.45, 129.68, 128.32, 127.23, 126.47, 57.84, 55.55, 52.58, 46.51, 41.59, 24.09; MS (ESI, m/z) calculated for C₁₄H₁₉ClNO [M+H]⁺ 252.12, found 252.05.

### 1-(4-Chlorobenzyl)-2,2-dimethylpiperidin-4-one (39)

The title compound was synthesized according to General Procedure B. Yield: 54.5%; ¹H NMR (400 MHz, chloroform-d) δ 7.35-7.27 (m, 4H), 3.59 (s, 2H), 2.73 (t, J=6.3 Hz, 2H), 2.38 (s, 2H), 2.36-2.29 (m, 2H), 1.18 (s, 6H); ¹³C NMR (100 MHz, chloroform-d) δ 210.03, 139.02, 132.63, 129.67, 128.57, 57.81, 55.56, 52.33, 46.34, 41.58, 24.08; MS (ESI, m/z) calculated for C₁₄H₁₉ClNO [M+H]⁺ 252.12, found 252.05.

### 1-Benzyl-2,2-dimethylpiperidin-4-amine (40)

The title compound was synthesized according to General Procedure B. Yield: 85.8%; ¹H NMR (400 MHz, Methanol-d4) δ 7.33-7.22 (m, 4H), 7.19 (t, J=7.1 Hz, 1H), 3.49 (dd, J=435.5, 13.5 Hz, 2H), 2.88-2.77 (m, 1H), 2.61-2.50 (m, 1H), 2.35-2.23 (m, 1H), 1.74-1.59 (m, 2H), 1.33 (t, J=12.2 Hz, 1H), 1.27 (d, J=3.6 Hz, 3H), 1.23-1.11 (m, 1H), 1.06 (s, 3H); ¹³C NMR (100 MHz, Methanol-d4) δ 141.75, 129.97, 129.13, 127.74, 55.37, 54.73, 50.65, 47.02, 46.60, 36.70, 31.21, 16.36; MS (ESI, m/z) calculated for C₁₄H₂₃N₂ [M+H]⁺ 219.19, found 219.15.

### 1-(3-Chlorobenzyl)-2,2-dimethylpiperidin-4-amine (41)

The title compound was synthesized according to General Procedure B. Yield: 98.0%; ¹H NMR (400 MHz, Methanol-d4) δ 7.37 (s, 1H), 7.28-7.23 (m, 2H), 7.23-7.19 (m, 1H), 3.51 (dd, J=422.1, 14.1 Hz, 2H), 2.90-2.81 (m, 1H), 2.58-2.49 (m, 1H), 2.40-2.30 (m, 1H), 1.78-1.71 (m, 1H), 1.71-1.65 (m, 1H), 1.35 (t, J=12.2 Hz, 1H), 1.26 (d, J=2.9 Hz, 3H), 1.23-1.16 (m, 1H), 1.07 (s, 3H); ¹³C NMR (100 MHz, Methanol-d4) δ 144.78, 135.13, 130.61, 129.45, 128.00, 127.75, 55.30, 54.17, 50.67, 47.23, 46.61, 36.81, 31.26, 16.44; MS (ESI, m/z) calculated for C₁₄H₂₂ClN₂ [M+H]⁺ 253.15, found 253.10.

### 1-(4-Chlorobenzyl)-2,2-dimethylpiperidin-4-amine (42)

The title compound was synthesized according to General Procedure B. Yield: 93.9%; ¹H NMR (400 MHz, Methanol-d4) δ 7.28 (q, J=8.7 Hz, 4H), 3.47 (dd, J=418.1, 13.9 Hz, 2H), 2.89-2.78 (m, 1H), 2.56-2.43 (m, 1H), 2.33-2.24 (m, 1H), 1.76-1.61 (m, 2H), 1.36-1.27 (m, 1H), 1.24 (s, 3H), 1.21-1.10 (m, 1H), 1.05 (s, 3H); ¹³C NMR (101 MHz, Methanol-d4) δ 140.91, 133.26, 131.22, 129.16, 55.29, 53.92, 50.62, 47.08, 46.59, 36.74, 31.25, 16.39; MS (ESI, m/z) calculated for C₁₄H₂₁ClN₂ [M+H]⁺ 253.15, found 253.10.

### General Procedure C

Triethylamine (1 eq), acetic acid (1.5 eq), and sodium triacetoxyborohydride (2 eq) were added to the Boc-deprotected intermediates 29-32 and an aldehyde solution (DCM (5 mL)). The mixture was stirred at 60° C. for at least 12 h. The resulting mixture was washed with an aqueous NaHCO₃ solution, diluted with ethyl acetate, washed with H₂O and brine, dried over MgSO₄, and purified by silica gel flash chromatography (dichloromethane:methanol=10:1 elution) to afford the title products 43-49, 56a, 56b, 57a, 57b, 58a, and 58b.

### 4-((trans-1-Benzyl-3-fluoropiperidin-4-yl)amino)-N-methyl-1H-pyrrolo[2,3-b]pyridine-5-carboxamide (43a)

The title compound was synthesized according to General Procedure C. Yield: 55.8%; ¹H NMR (400 MHz, chloroform-d) δ 11.20 (s, 1H), 9.36 (d, J=8.2 Hz, 1H), 8.25 (d, J=3.8 Hz, 1H), 7.34 (s, 1H), 7.33 (s, 2H), 7.32-7.23 (m, 2H), 7.05 (d, J=3.6 Hz, 1H), 6.59 (d, J=3.5 Hz, 1H), 6.31-6.17 (m, 1H), 4.78-4.54 (m, 1H), 4.32-4.17 (m, 1H), 3.60 (s, 2H), 3.11-3.04 (m, 1H), 2.98 (d, J=4.7 Hz, 3H), 2.80-2.71 (m, 1H), 2.54-2.42 (m, 1H), 2.37 (t, J=9.5 Hz, 1H), 2.31-2.22 (m, 1H), 1.80-1.67 (m, 1H); ¹³C NMR (100 MHz, chloroform-d) δ 170.82, 150.67, 150.25, 143.38, 137.82, 128.98, 128.36, 127.27, 121.42, 105.41, 103.95, 102.48, 90.63 (d, J_{C-F}=180.1 Hz), 62.42, 55.43 (d, J_{C-F}=23.6 Hz), 53.96 (d, J_{C-F}=23.0 Hz), 50.46, 30.17, 26.66; HRMS (ESI, m/z) calculated C₂₁H₂₅FN₅O [M+H]⁺ 382.2038, found 382.2041.

### 4-((trans-1-(4-Chlorobenzyl)-3-fluoropiperidin-4-yl)amino)-N-methyl-1H-pyrrolo[2,3-b]pyridine-5-carboxamide (43b)

The title compound was synthesized according to General Procedure C. Yield: 65.6%; ¹H NMR (400 MHz, chloroform-d) δ 11.37 (s, 1H), 9.38 (d, J=8.2 Hz, 1H), 8.25 (d, J=4.1 Hz, 1H), 7.34 7.21 (m, 4H), 7.04 (d, J=3.1 Hz, 1H), 6.57 (d, J=3.4 Hz, 1H), 6.30 (s, 1H), 4.74-4.55 (m, 1H), 4.32-4.19 (m, 1H), 3.55 (s, 2H), 3.02 (d, J=14.5 Hz, 1H), 2.98 (d, J=4.7 Hz, 3H), 2.75-2.67 (m, 1H), 2.56-2.43 (m, 1H), 2.37 (t, J =9.0 Hz, 1H), 2.33-2.21 (m, 1H), 1.81-1.67 (m, 1H); ¹³C NMR (100 MHz, chloroform-d) δ 170.78, 150.23, 143.23, 143.16, 136.40, 132.97, 130.19, 128.52, 121.54, 105.43, 103.91, 102.37, 90.37 (d, J_{C-F}=179.6 Hz), 61.59, 55.22 (d, J_{C-F}=23.8 Hz), 53.61 (d, J_{C-F}=24.1 Hz), 50.23, 29.95, 26.66; HRMS (ESI, m/z) calculated for C₂₁H₂₄ClFN₅O₂ [M+H]⁺ 416.1648, found 416.1651.

### 4-((trans-1-(3-Methoxybenzyl)-3-fluoropiperidin-4-yl)amino)-N-methyl-1H-pyrrolo[2,3-b]pyridine-5-carboxamide (43c)

The title compound was synthesized according to General Procedure C. Yield: 61.3%; ¹H NMR (400 MHz, chloroform-d) δ 11.40 (s, 1H), 9.36 (d, J=8.2 Hz, 1H), 8.24 (d, J=5.6 Hz, 1H), 7.28-7.17 (m, 1H), 7.08-6.96 (m, 1H), 6.94-6.87 (m, 2H), 6.81 (dd, J=8.9, 1.8 Hz, 1H), 6.56 (d, J=3.0 Hz, 1H), 6.30 (s, 1H), 4.77-4.55 (m, 1H), 4.34-4.15 (m, 1H), 3.81 (s, 3H), 3.57 (s, 2H), 3.10 (td, J=15.5, 13.4, 6.8 Hz, 1H), 2.97 (d, J=4.7 Hz, 3H), 2.82-2.75 (m, 1H), 2.47 (q, J=7.1 Hz, 1H), 2.35 (t, J=9.5 Hz, 1H), 2.26 (d, J=5.3 Hz, 1H), 1.85-1.68 (m, 1H); ¹³C NMR (100 MHz, chloroform-d) δ 170.79, 159.75, 150.30, 143.17, 143.02, 139.55, 129.31, 121.50, 121.23, 114.21, 112.85, 105.42, 103.94, 102.41, 90.68 (d, J_{C-F}=180.1 Hz), 62.31, 55.50 (d, J_{C-F}=24.1 Hz), 55.23, 54.16 (d, J_{C-F}=22.8 Hz), 50.53, 30.27, 26.65; HRMS (ESI, m/z) calculated for C₂₂H₂₇FN₅O₂ [M+H]⁺ 412.2143, found 412.2139.

### 4-((trans-1-(4-Methoxybenzy0-3-fluoropiperidin-4-yl)amino)-N-methyl-1H-pyrrolo[2,3-b]pyridine-5-carboxamide (43d)

The title compound was synthesized according to General Procedure C. Yield: 69.2%; ¹H NMR (400 MHz, chloroform-d) δ 11.23 (s, 1H), 9.35 (d, J=8.2 Hz, 1H), 8.24 (d, J=4.1 Hz, 1H), 7.23 (d, J=8.5 Hz, 2H), 7.06-6.97 (m, 1H), 6.87 (d, J=8.6 Hz, 2H), 6.56 (d, J=3.2 Hz, 1H), 6.34 (s, 1H), 4.74-4.54 (m, 1H), 4.26-4.08 (m, 1H), 3.80 (s, 3H), 3.53 (s, 2H), 3.07 (d, J=5.1 Hz, 1H), 2.97 (d, J=4.7 Hz, 3H), 2.79-2.69 (m, 1H), 2.44 (d, J=10.5 Hz, 1H), 2.33 (t, J=9.4 Hz, 1H), 2.29-2.19 (m, 1H), 1.79-1.66 (m, 1H); ¹³C NMR (100 MHz, chloroform-d) δ 170.78, 150.23, 143.23, 143.16, 136.40, 132.97, 130.19, 128.52, 121.54, 105.43, 103.91, 102.37, 90.37 (d, J_{C-F}=178.4 Hz), 61.59, 60.43, 55.22 (d, J_{C-F}=23.1 Hz), 53.61 (d, J_{C-F}=17.6 Hz), 50.23, 29.95, 26.66; HRMS (ESI, m/z) calculated for C₂₂H₂₇FN₅O [M+H]⁺ 412.2143, found 412.2146.

### 4-((trans-1-(3-Cyanobenzyl)-3-fluoropiperidin-4-yl)amino)-N-methyl-1H-pyrrolo[2,3-b]pyridine-5-carboxamide (43e)

The title compound was synthesized according to General Procedure C. Yield: 59.1%; ¹H NMR (400 MHz, chloroform-d) δ 11.19 (s, 1H), 9.42 (d, J=8.2 Hz, 1H), 8.27 (s, 1H), 7.64 (s, 1H), 7.62-7.52 (m, 2H), 7.44 (t, J=7.7 Hz, 1H), 7.09 (d, J=3.6 Hz, 1H), 6.60 (d, J=3.7 Hz, 1H), 6.28 (d, J=4.7 Hz, 1H), 4.78-4.55 (m, 1H), 4.34-4.25 (m, 1H), 3.61 (s, 2H), 2.99 (d, J=4.7 Hz, 3H), 3.03-2.90 (m, 1H), 2.71 (s, 1H), 2.61-2.50 (m, 1H), 2.43 (t, J=8.7 Hz, 1H), 2.36-2.25 (m, 1H), 1.84-1.73 (m, 1H); ¹³C NMR (100 MHz, chloroform-d) δ 170.91, 150.79, 150.25, 143.52, 139.82, 133.28, 132.32, 131.17, 129.34, 121.68, 119.00, 112.60, 105.55, 104.04, 102.48, 90.09 (d, J_{C-F}=180.0 Hz), 60.53, 55.14 (d, J_{C-F}=22.5 Hz), 53.19 (d, J_{C-F}=18.6 Hz), 50.16, 29.81, 26.78; HRMS (ESI, m/z) calculated for C₂₂H₂₄FN₆O [M+H]⁺ 407.1990, found 407.1995.

### 4-((trans-1-(4-Cyanobenzyl)-3-fluoropiperidin-4-yl)amino)-N-methyl-1H-pyrrolo[2,3-b]pyridine-5-carboxamide (43f)

The title compound was synthesized according to General Procedure C. Yield: 55.8%; ¹H NMR (400 MHz, chloroform-d) δ 11.40 (s, 1H), 9.43 (d, J=8.2 Hz, 1H), 8.27 (s, 1H), 7.62 (d, J=8.2 Hz, 2H), 7.45 (d, J=8.1 Hz, 2H), 7.07 (d, J=3.5 Hz, 1H), 6.58 (d, J=3.4 Hz, 1H), 6.32 (s, 1H), 4.79-4.54 (m, 1H), 4.28 (d, J=4.8 Hz, 1H), 3.63 (s, 2H), 3.10-2.91 (m, 1H), 2.99 (d, J=4.7 Hz, 3H), 2.70 (s, 1H), 2.61-2.49 (m, 1H), 2.42 (t, J=8.7 Hz, 1H), 2.37-2.23 (m, 1H), 1.84-1.70 (m, 1H); ¹³C NMR (100 MHz, Chloroform-d) δ 170.88, 150.61, 150.28, 143.88, 143.33, 132.35, 129.37, 121.75, 119.02, 111.19, 105.56, 103.99, 102.40, 90.12 (d, J_{C-F}=179.6 Hz), 61.86, 55.19 (d, J_{C-F}=23.6 Hz), 53.17 (d, J_{C-F}=25.0 Hz), 50.24, 29.80, 26.77; HRMS (ESI, m/z) calculated for C₂₂H₂₄FN₆O [M+H]⁺ 407.1990, found 407.1996.

### 4-((cis-1-Benzyl-3-fluoropiperidin-4-yl)amino)-N-methyl-1H-pyrrolo[2,3-b]pyridine-5-carboxamide (44a)

The title compound was synthesized according to General Procedure C. Yield: 26.0%; ¹H NMR (400 MHz, chloroform-d) δ 10.99 (s, 1H), 9.45 (d, J=8.4 Hz, 1H), 8.25 (s, 1H), 7.37-7.31 (m, 4H), 7.29-7.26 (m, 1H), 7.07 (d, J=3.6 Hz, 1H), 6.44 (d, J=3.7 Hz, 1H), 6.15 (d, J=4.6 Hz, 1H), 4.87 (d, J=48.3 Hz, 1H), 4.33-4.05 (m, 1H), 3.63 (s, 2H), 3.20 (s, 1H), 2.99 (d, J=4.8 Hz, 3H), 2.91 (d, J=10.0 Hz, 1H), 2.54-2.37 (m, 1H), 2.32 (t, J=10.6 Hz, 1H), 2.16-2.05 (m, 1H), 2.05-1.98 (m, 1H); ¹³C NMR (100 MHz), chloroform-d) δ 170.82, 150.67, 150.25, 143.38, 137.82, 128.98, 128.36, 127.27, 121.42, 105.41, 103.95, 102.48, 88.25 (d, J_{C-F}=180.1 Hz), 62.42, 55.13 (d, J_{C-F}=22.2 Hz), 52.53 (d, J_{C-F}=19.8 Hz), 50.46, 30.17, 26.66; HRMS (ESI, m/z) calculated C₂₁H₂₅FN₅O [M+H]⁺ 382.2038, found 382.2040.

### 4-((cis-1-(3-Chlorobenzyl)-3-fluoropiperidin-4-yl)amino)-N-methyl-1H-pyrrolo[2,3-b]pyridine-5-carboxamide (44b)

The title compound was synthesized according to General Procedure C. Yield: 35.1%; ¹H NMR (400 MHz, DMSO-d6) δ 12.32 (s, 1H), 10.42 (d, J=8.6 Hz, 1H), 9.11 (s, 1H), 9.04 (d, J=4.5 Hz, 1H), 8.22-8.14 (m, 2H), 8.14-8.04 (m, 2H), 7.96 (d, J=2.1 Hz, 1H), 7.30 (d, J=3.0 Hz, 1H), 5.64 (d, J=49.5 Hz, 1H), 5.10-4.88 (m, 1H), 4.37 (s, 2H), 3.86 (s, 1H), 3.58 (s, 1H), 3.54 (d, J=4.3 Hz, 3H), 3.23 (d, J=12.6 Hz, 1H), 3.12 (t, J=10.7 Hz, 1H), 2.60 (t, J=10.7 Hz, 2H); ¹³C NMR (100 MHz, DMSO-d6) δ 169.97, 150.33, 148.69, 144.47, 140.85, 133.00, 130.12, 128.36, 127.36, 126.98, 122.14, 104.65, 103.23, 101.42, 88.59 (d, J_{C-F}=177.1 Hz), 60.36, 54.58 (d, J_{C-F}=17.6 Hz), 51.33 (d, J_{C-F}=17.9 Hz), 50.14, 27.95, 26.09; HRMS (ESI, m/z) calculated for C₂₁H₂₄ClFN₅O [M+H]⁺ 416.1648, found 416.1648.

### 4-((cis-1-(4-Chlorobenzyl)-3-fluoropiperidin-4-yl)amino)-N-methyl-1H-pyrrolo[2,3-b]pyridine-5-carboxamide (44c)

The title compound was synthesized according to General Procedure C. Yield: 35.1%; ¹H NMR (400 MHz, DMSO-d₆) δ 11.52 (s, 1H), 9.63 (d, J=8.7 Hz, 1H), 8.32 (s, 1H), 8.27-8.17 (m, 1H), 7.44-7.30 (m, 4H), 7.17 (dd, J=3.4, 2.4 Hz, 1H), 6.51 (d, J=2.1 Hz, 1H), 4.85 (d, J=49.4 Hz, 1H), 4.29-4.14 (m, 1H), 3.55 (s, 2H), 3.05 (d, J=9.3 Hz, 1H), 2.81-2.77 (m, 1H), 2.75 (d, J=4.4 Hz, 3H), 2.43 (d, J=12.7 Hz, 1H), 2.31 (t, J=11.1 Hz, 1H), 1.90 (d, J=9.1 Hz, 1H), 1.79 (q, J=10.7 Hz, 1H); ¹³C NMR (100 MHz, DMSO-d₆) δ 170.42, 150.78, 149.15, 144.92, 137.59, 131.97, 131.00, 128.64, 122.56, 105.08, 103.66, 101.86, 89.03 (d, J_{C-F}=177.2 Hz), 60.77, 55.03 (d, J_{C-F}=20.5 Hz), 51.82 (d, J_{C-F}=17.6 Hz), 33.91, 28.37, 26.5; HRMS (ESI, m/z) calculated for C₂₁H₂₄ClFN₅O [M+H]⁺ 416.1648, found 416.1648.

### 4-((cis-1-(4-Methoxybenzyl)-3-fluoropiperidin-4-yl)amino)-N-methyl-1H-pyrrolo[2,3-b]pyridine-5-carboxamide (44d)

The title compound was synthesized according to General Procedure C. Yield: 50.3%; ¹H NMR (400 MHz, DMSO-d6) 6 11.52 (s, 1H), 9.62 (d, J=8.7 Hz, 1H), 8.31 (s, 1H), 8.27-8.19 (m, 1H), 7.23 (d, J=8.7 Hz, 2H), 7.16 (dd, J=3.3, 2.2 Hz, 1H), 6.95-6.83 (m, 2H), 6.49 (d, J=2.5 Hz, 1H), 4.83 (d, J=49.6 Hz, 1H), 4.26-4.09 (m, 1H), 3.74 (s, 3H), 3.48 (s, 2H), 3.04 (s, 1H), 2.82-2.77 (m, 1H), 2.74 (d, J=4.4 Hz, 3H), 2.48-2.31 (m, 1H), 2.26 (t, J=10.8 Hz, 1H), 1.89 (d, J=9.9 Hz, 1H), 1.76 (q, J=10.9 Hz, 1H); ¹³C NMR (100 MHz, DMF-d6) δ 169.97, 158.33, 150.32, 148.71, 144.47, 130.06, 129.74, 122.11, 113.59, 104.62, 103.19, 101.41, 88.62 (d, J_{C-F}=177.0 Hz), 60.73, 54.99, 54.53 (d, J_{C-F}=14.5 Hz), 51.48 (d, J_{C-F}=17.5 Hz), 50.05, 27.95, 26.0; HRMS (ESI, m/z) calculated for C₂₂H₂₇FN₅O₂ [M+H]⁺ 412.2143, found 412.2148.

### 4-((cis-1-(3-Cyanobenzyl)-3-fluoropiperidin-4-yl)amino)-N-methyl-1H-pyrrolo[2,3-b] pyridine-5-carboxamide (44e)

The title compound was synthesized according to General Procedure C. Yield: 49.1%; ¹H NMR (400 MHz, DMSO-d₆) δ 11.52 (s, 1H), 9.63 (d, J=8.7 Hz, 1H), 8.32 (s, 1H), 8.24 (d, J=4.5 Hz, 1H), 7.78-7.72 (m, 2H), 7.69 (d, J=7.9 Hz, 1H), 7.57 (t, J=7.7 Hz, 1H), 7.17 (dd, J=3.2, 2.2 Hz, 1H), 6.51 (d, J=2.4 Hz, 1H), 4.86 (d, J=49.4 Hz, 1H), 4.30-4.14 (m, 1H), 3.63 (s, 2H), 3.06 (t, J=9.3 Hz, 1H), 2.81-2.77 (m, 1H), 2.75 (d, J=4.4 Hz, 3H), 2.51 (dd, J=35.7, 12.9 Hz, 1H), 2.34 (t, J=10.6 Hz, 1H), 1.90 (d, J=9.7 Hz, 1H), 1.80 (q, J=10.7 Hz, 1H); ¹³C NMR (101 MHz, DMSO-d₆) δ 169.96, 150.32, 148.69, 144.46, 139.93, 133.64, 132.09, 130.90, 129.53, 122.13, 118.93, 111.24, 104.65, 103.24, 101.43, 88.58 (d, J_{C-F}=176.9 Hz), 60.02, 54.50 (d, J_{C-F}=18.4 Hz), 51.28 (d, J_{C-F}=17.9 Hz), 50.04, 27.91, 26.08; HRMS (ESI, m/z) calculated for C₂₂H₂₄FN₆O [M+H]⁺ 407.1990, found 407.1991.

### 4-((cis-1-(4-Cyanobenzyl)-3-fluoropiperidin-4-yl)amino)-N-methyl-1H-pyrrolo[2,3-b]pyridine-5-carboxamide (44f)

The title compound was synthesized according to General Procedure C. Yield: 31.9%; ¹H NMR (400 MHz, DMSO-d₆) δ 11.52 (s, 1H), 9.62 (d, J=8.7 Hz, 1H), 8.31 (s, 1H), 8.24 (d, J=4.6 Hz, 1H), 7.82 (d, J=8.3 Hz, 2H), 7.54 (d, J=8.3 Hz, 2H), 7.17 (dd, J=3.3, 2.4 Hz, 1H), 6.52-6.49 (m, 1H), 4.85 (d, J=49.5 Hz, 1H), 4.29-4.14 (m, 1H), 3.66 (s, 2H), 3.06 (t, J=9.3 Hz, 1H), 2.82-2.76 (m, 1H), 2.74 (d, J=4.4 Hz, 3H), 2.60-2.42 (m, 1H), 2.35 (t, J=10.4 Hz, 1H), 1.90 (d, J=9.8 Hz, 1H), 1.85-1.71 (m, 1H); ¹³C NMR (100 MHz, DMSO-d₆) δ 169.96, 150.32, 148.68, 144.46, 144.34, 132.23, 129.48, 122.15, 118.95, 109.77, 104.64, 103.23, 101.41, 88.57 (d, J_{C-F}=177.1 Hz), 60.50, 54.65 (d, J_{C-F}=19.7 Hz), 51.27 (d, J_{C-F}=18.0 Hz), 50.17, 27.92, 26.08; HRMS (ESI, m/z) calculated for C₂₂H₂₄FN₆O [M+H]+407.1990, found 407.1991.

### 4-((1-Benzyl-3,3-difluoropiperidin-4-y0amino)-N-methyl-1H-pyrrolo[2,3-b]pyridine-5-carboxamide (45a)

The title compound was synthesized according to General Procedure C. Yield: 36.5%; ¹H NMR (400 MHz, chloroform-d) δ 12.07 (s, 1H), 8.98 (s, 1H), 8.63 (d, J=4.5 Hz, 1H), 7.41-7.33 (m, 4H), 7.33-7.28 (m, 1H), 6.64 (d, J=3.4 Hz, 1H), 4.01 (s, 2H), 3.94-3.84 (m, 1H), 3.58-3.45 (m, 2H), 3.24-3.08 (m, 2H), 3.02 (d, J=4.8 Hz, 3H), 2.13 (dd, J=8.5, 3.9 Hz, 1H), 1.88-1.76 (m, 1H); ¹³C NMR (100 MHz, chloroform-d) δ 167.24, 151.70, 149.17, 146.66, 139.73, 128.60, 128.08, 127.32, 125.47, 120.7 (t, J_{C-F}=247.02 Hz), 116.52, 114.42, 100.67, 62.25, 56.42 (t, J_{C-F}=21.4 Hz) 55.01 (t, J_{C-F}=29.4 Hz), 51.59, 30.95, 29.41, 26.29; HRMS (ESI, m/z) calculated for C₂₁H₂₄F₂N₅O [M+H]⁺ 400.1943, found 400.1946.

### 4-41-(3-Chlorobenzyl)-3,3-difluoropiperidin-4-yl)amino)-N-methyl-1H-pyrrolo[2,3-b]pyridine-5-carboxamide (45b)

The title compound was synthesized according to General Procedure C. Yield: 60.6%; ¹H NMR (400 MHz, chloroform-d) δ 12.30 (s, 1H), 8.96 (s, 1H), 8.66-8.51 (m, 1H), 7.41 (s, 1H), 7.38 (d, J=3.0 Hz, 1H), 7.30-7.24 (m, 3H), 6.63 (d, J=3.3 Hz, 1H), 4.00 (d, J=7.1 Hz, 2H), 3.93-3.82 (m, 1H), 3.59-3.44 (m, 2H), 3.22 (t, J=10.0 Hz, 1H), 3.17-3.06 (m, 1H), 3.03 (d, J=4.8 Hz, 3H), 2.13 (dd, J=9.0, 4.5 Hz, 1H), 1.88-1.77 (m, 1H); ¹³C NMR (100 MHz, chloroform-d) δ 167.38, 151.80, 149.20, 146.61, 142.09, 134.54, 129.94, 128.24, 127.56, 126.25, 125.70, 120.74 (t, J_{C-F}=246.2 Hz), 116.56, 114.54, 100.69, 60.36, 56.73 (t, J_{C-F}=21.5 Hz), 55.25 (t, J_{C-F}=29.0 Hz), 51.21, 31.07, 29.80, 26.42; HRMS (ESI, m/z) calculated for C₂₁H₂₃ClF₂N₅O [M+H]⁺ 434.1554, found 434.1559.

### 4-41-(4-Chlorobenzyl)-3,3-difluoropiperidin-4-yl)amino)-N-methyl-1H-pyrrolo[2,3-b]pyridine-5-carboxamide (45c)

The title compound was synthesized according to General Procedure C. Yield: 60.6%; ¹H NMR (400 MHz, Methanol-d4) δ 9.84 (s, 1H), 8.91 (dd, J=23.9, 8.5 Hz, 4H), 8.86 (s, 1H), 8.23 (d, J=3.7 Hz, 1H), 5.50 (s, 2H), 5.46-5.35 (m, 2H), 5.25-5.15 (m, 1H), 5.11-4.98 (m, 1H), 4.69-4.57 (m, 1H), 4.50 (s, 3H), 3.72-3.63 (m, 1H), 3.47-3.34 (m, 1H); ¹³C NMR (100 MHz, Methanol-d₄) δ 171.48, 151.58, 150.61, 145.66, 140.08, 133.83, 130.96, 129.50, 125.90, 121.8 (t, J_{C-F}=247.3 Hz), 117.01, 114.62, 101.72, 60.77, 58.0 (t, J_{C-F}=20.8 Hz), 56.2 (t, J_{C-F}=29.7 Hz), 51.58, 50.14, 30.46, 26.85; HRMS (ESI, m/z) calculated for C₂₁H₂₃ClF₂N₅O [M+H]⁺ 434.1554, found 434.11557.

### 4-41-(3-Methoxybenzyl)-3,3-difluoropiperidin-4-ypamino)-N-methyl-1H-pyrrolo[2,3-b]pyridine-5-carboxamide (45d)

The title compound was synthesized according to General Procedure C. Yield: 60.6%; ¹H NMR (400 MHz, chloroform-d) δ 11.72 (s, 1H), 8.98 (s, 1H), 8.60 (s, 1H), 7.38 (s, 1H), 7.28 (d, J=10.4 Hz, 1H), 6.97 (d, J=6.7 Hz, 2H), 6.84 (d, J=8.4 Hz, 1H), 6.65 (s, 1H), 3.99 (s, 2H), 3.95-3.87 (m, 1H), 3.84 (s, 3H), 3.51 (t, J=14.6 Hz, 2H), 3.21 (t, J=10.7 Hz, 1H), 3.14 (d, J=5.7 Hz, 25 1H), 3.03 (d, J=4.3 Hz, 3H), 2.12 (s, 1H), 1.95-1.79 (m, 1H); ¹³C NMR (100 MHz, chloroform-d) δ 167.20, 159.85, 151.63, 149.15, 146.77, 141.38, 129.61, 125.35, 120.63 (t, J_{C-F}=246.2 Hz), 120.31, 116.58, 114.36, 113.67, 112.57, 100.75, 56.32 (t, J_{C-F}=21.5 Hz), 55.24, 55.03 (t, J_{C-F}=29.0 Hz), 51.44, 50.39, 29.41, 26.29; HRMS (ESI, m/z) calculated for C₂₂H₂₆F₂N₅O [M+H]⁺ 430.2049, found 430.2057.

### 4-41-(4-Methoxybenzyl)-3,3-difluoropiperidin-4-yl)amino)-N-methyl-1H-pyrrolo[2,3-b]pyridine-5-carboxamide (45e)

The title compound was synthesized according to General Procedure C. Yield: 62.3%; ¹H NMR (400 MHz, chloroform-d) δ 11.83 (s, 1H), 8.98 (s, 1H), 8.61 (d, J=4.7 Hz, 1H), 7.38 (d, J=2.5 Hz, 1H), 7.30 (d, J=8.6 Hz, 2H), 6.95-6.87 (m, 2H), 6.64 (d, J=3.4 Hz, 1H), 3.94 (s, 2H), 3.91-3.84 (m, 1H), 3.82 (s, 3H), 3.55-3.49 (m, 2H), 3.20 (t, J=10.0 Hz, 1H), 3.16-3.06 (m, 1H), 3.02 (d, J=4.8 Hz, 3H), 2.11 (dd, J=8.2, 4.2 Hz, 1H), 1.88-1.78 (m, 1H); ¹³C NMR (100 MHz, chloroform-d) δ 167.24, 158.90, 151.73, 149.19, 146.80, 131.81, 129.31, 125.38, 120.68 (t, J_{C-F}=247.0 Hz), 116.63, 114.41, 113.99, 100.75, 56.30 (t, J_{C-F}=21.5 Hz), 55.32, 55.04 (t, J_{C-F}=28.9 Hz), 51.02, 50.35, 29.46, 26.28; HRMS (ESI, m/z) calculated for C₂₂H₂₆F₂N₅O [M+H]⁺ 430.2049, found 430.2052.

### 4-41-(3-Cyanobenzyl)-3,3-difluoropiperidin-4-yl)amino)-N-methyl-1H-pyrrolo[2,3-b]pyridine-5-carboxamide (45)

The title compound was synthesized according to General Procedure C. Yield: 30.3%; ¹H NMR (400 MHz, chloroform-d) δ 9.33 (s, 1H), 8.96 (s, 1H), 8.45 (s, 1H), 8.17 (t, J=1.4 Hz, 1H), 8.08-8.00 (m, 1H), 7.82-7.75 (m, 1H), 7.60 (d, J=7.9 Hz, 1H), 7.36 (dd, J=3.6, 2.5 Hz, 1H), 6.75 (dd, J=3.7, 2.0 Hz, 1H), 4.07 (d, J=11.2 Hz, 1H), 3.88 (d, J=10.1 Hz, 2H), 3.69-3.59 (m, 1H), 3.55-3.47 (m, 1H), 3.35-3.27 (m, 1H), 3.07 (d, J=4.0 Hz, 3H), 2.03 (d, J=11.0 Hz, 1H), 1.36-1.26 (m, 2H); HRMS (ESI, m/z) calculated for C₂₂H₂₃F₂N₆O [M+H]⁺ 425.1896, found 425.1898.

### 4-41-(4-Cyanobenzyl)-3,3-difluoropiperidin-4-yl)amino)-N-methyl-1H-pyrrolo[2,3-b]pyridine-5-carboxamide (45g)

The title compound was synthesized according to General Procedure C. Yield: 58.6%; ¹H NMR (400 MHz, chloroform-d) δ 11.64 (s, 1H), 8.94 (s, 1H), 8.43 (d, J=4.5 Hz, 1H), 7.68-7.65 (m, 2H), 7.53 (d, J=8.4 Hz, 2H), 7.38 (d, J=3.2 Hz, 1H), 6.63 (d, J=3.4 Hz, 1H), 4.18-4.05 (m, 2H), 3.94-3.84 (m, 1H), 3.55-3.47 (m, 2H), 3.25 (t, J=10.4 Hz, 1H), 3.11-3.05 (m, 1H), 3.03 (d, J=4.8 Hz, 3H), 2.17-2.12 (m, 1H), 1.88-1.78 (m, 1H); HRMS (ESI, m/z) calculated for C₂₂H₂₃F₂N₆O [M+H]⁺ 425.1896, found 425.1897.

### 4-((cis-1-(2-Fluorobenzyl)-2-methylpiperidin-4-yl)amino)-N-methyl-1H-pyrrolo[2,3-b]pyridine-5-carboxamide (46a)

The title compound was synthesized according to General Procedure C. Yield: 46.7%; ¹H NMR (400 MHz, chloroform-d) δ 9.10 (d, J=7.8 Hz, 1H), 8.22 (s, 1H), 7.47-7.39 (m, 1H), 7.26-7.20 (m, 1H), 7.15-7.10 (m, 1H), 7.07-7.00 (m, 2H), 6.50 (d, J=3.7 Hz, 1H), 6.30 (s, 1H), 4.05 (d, J=13.9 Hz, 1H), 4.01-3.87 (m, 1H), 3.38 (d, J=13.8 Hz, 1H), 2.96 (d, J=4.7 Hz, 3H), 2.93 (d, J=3.2 Hz, 1H), 2.52-2.41 (m, 1H), 2.21-2.06 (m, 3H), 1.66-1.44 (m, 2H), 1.26 (d, J=6.1 Hz, 3H); ¹³C NMR (100 MHz, chloroform-d) δ 170.70, 161.37 (d, J_{C-F}=245.5 Hz), 150.05, 149.90, 143.24, 131.36 (d, J_{C-F}=4.6 Hz), 128.4 (d, J_{C-F}=8.2 Hz), 125.83 (d, J_{C-F}=14.6 Hz), 123.88 (d, J_{C-F}=3.4 Hz), 115.18 (d, J_{C-F}=22.4 Hz), 105.15, 103.56, 102.75, 55.93, 51.48, 51.22, 49.88, 42.37, 33.47, 26.60, 21.17; HRMS (ESI, m/z) calculated for C₂₂H₂₇FN₅O [M+H]⁺ 396.2194, found 369.2205.

### 4-((cis-1-(3-Fluorobenzyl)-2-methylpiperidin-4-yl)amino)-N-methyl-1H-pyrrolo[2,3-b]pyridine-5-carboxamide (46b)

The title compound was synthesized according to General Procedure C. Yield: ¹H NMR (400 MHz, chloroform-d) δ 9.24 (d, J=7.6 Hz, 1H), 8.18 (s, 1H), 7.31-7.24 (m, 1H), 7.09 (t, J=8.3 Hz, 2H), 7.01 (d, J=3.3 Hz, 1H), 6.97-6.90 (m, 1H), 6.48 (d, J=3.4 Hz, 1H), 6.41 (s, 1H), 4.01-3.88 (m, 1H), 3.63 (dd, J=383.8, 13.7 Hz, 2H), 2.96 (d, J=4.5 Hz, 3H), 2.89 (d, J=12.0 Hz, 1H), 2.55-2.39 (m, 1H), 2.15 (d, J=15.7 Hz, 1H), 2.08 (d, J=10.9 Hz, 1H), 1.70-1.45 (m, 2H), 1.23 (d, J=6.0 Hz, 3H); ¹³C NMR (100 MHz), chloroform-d) δ 170.28, 162.97 (d, J_{C-F}=245.5 Hz), 150.22, 148.35, 142.22 (d, J_{C-F}=7.0 Hz), 141.66, 129.60 (d, J_{C-F}=8.3 Hz), 124.28 (d, J_{C-F}=2.5 Hz), 121.38, 115.52 (d, J_{C-F}=21.2 Hz), 113.72 (d, J_{C-F}=21.1 Hz), 105.29, 103.59, 102.82, 57.08, 55.94, 51.63, 51.31, 42.22, 33.38, 26.62, 21.18; HRMS (ESI, m/z) calculated for C₂₂H₂₇FN₅O [M+H]⁺ 396.2194, found 369.2206.

### 4-((cis-1-(4-Fluorobenzyl)-2-methylpiperidin-4-yl)amino)-N-methyl-1H-pyrrolo[2,3-b]pyridine-5-carboxamide (46c)

The title compound was synthesized according to General Procedure C. Yield: 61.4%; ¹H NMR (400 MHz, chloroform-d) δ 9.13 (d, J=7.9 Hz, 1H), 8.22 (s, 1H), 7.36-7.23 (m, 2H), 7.04-6.97 (m, 3H), 6.48 (d, J=3.7 Hz, 1H), 6.41 (s, 1H), 4.08 (d, J=13.5 Hz, 1H), 3.98-3.82 (m, 1H), 3.12 (d, J=13.5 Hz, 1H), 2.96 (d, J=4.7 Hz, 3H), 2.90-2.80 (m, 1H), 2.46-2.35 (m, 1H), 2.15 (dd, J=12.7, 2.4 Hz, 1H), 2.06 (t, J=11.6 Hz, 2H), 1.63-1.44 (m, 2H), 1.25 (d, J=5.0 Hz, 3H); ¹³C NMR (100 MHz, chloroform-d) δ 170.59, 161.89 (d, J_{C-F}=244.5 Hz), 149.98, 149.42, 142.91, 134.82 (d, J_{C-F}=3.1 Hz), 130.36 (d, J_{C-F}=7.8 Hz), 121.15, 115.00 (d, J_{C-F}=21.2 Hz), 105.16, 103.58, 102.74, 58.36, 56.74, 55.93, 51.39, 42.30, 33.43, 26.60, 21.20; HRMS (ESI, m/z) calculated for C₂₂H₂₇FN₅O [M+H]⁺ 396.2194, found 369.2203.

### N-Methyl-4-((cis-2-methyl-1-(4-(trifluoromethyl)benzyl)piperidin-4-yl)amino)-1H-pyrrolo[2,3-b]pyridine-5-carboxamide (46d)

The title compound was synthesized according to General Procedure C. Yield: 36.5%; ¹H NMR (400 MHz, chloroform-d) δ 9.20 (d, J=7.8 Hz, 1H), 8.23 (s, 1H), 7.58 (d, J=8.1 Hz, 2H), 7.46 (d, J=8.1 Hz, 2H), 7.00 (d, J=3.3 Hz, 1H), 6.48 (d, J=3.5 Hz, 1H), 4.00-3.87 (m, 1H), 3.68 (dd, J=393.8), 14.0 Hz, 2H), 2.98 (d, J=4.6 Hz, 3H), 2.90-2.82 (m, 1H), 2.52-2.39 (m, 1H), 2.19-2.04 (m, 3H), 1.66-1.48 (m, 2H), 1.22 (d, J=6.1 Hz, 3H); ¹³C NMR (100 MHz, chloroform-d) δ 170.41, 150.37, 148.43, 143.99, 141.65, 129.02, 128.70 (q, J_{C-F}=32.1 Hz), 124.73 (q, J_{C-F}=3.5 Hz), 123.85 (q, J_{C-F}=273 Hz), 121.61, 105.46, 103.69, 102.89, 57.29, 56.23, 51.87, 51.41, 42.34, 33.50, 26.75, 21.35; HRMS (ESI, m/z) calculated for C₂₃H₂₇F₃N₅O [M+H]⁺ 446.2162, found 446.2160.

### 4-((cis-14(5-Chloropyridin-2-yl)methyl)-2-methylpiperidin-4-yl)amino)-N-methyl-1H-pyrrolo[2,3-b]pyridine-5-carboxamide (47a)

The title compound was synthesized according to General Procedure C. Yield: 40.5%; ¹H NMR (400 MHz, chloroform-d) δ 9.16 (d, J=7.9 Hz, 1H), 8.49 (d, J=2.1 Hz, 1H), 8.23 (s, 1H), 7.65 (dd, J=8.4, 2.5 Hz, 1H), 7.46 (d, J=8.4 Hz, 1H), 7.02 (d, J=3.6 Hz, 1H), 6.51 (s, 1H), 6.49 (d, J=3.7 Hz, 1H), 4.01-3.89 (m, 1H), 3.77 (dd, J=296.5, 14.7 Hz, 2H), 2.96 (d, J=4.7 Hz, 3H), 2.91-2.84 (m, 1H), 2.55-2.43 (m, 1H), 2.32-2.23 (m, 1H), 2.21-2.06 (m, 2H), 1.70-1.44 (m, 2H), 1.19 (d, J=6.1 Hz, 3H); ¹³C NMR (100 MHz, chloroform-d) δ 170.60, 158.42, 149.99, 149.33, 147.74, 142.87, 136.24, 130.16, 123.77, 121.27, 105.20, 103.59, 102.71, 58.88, 56.20, 52.44, 51.19, 42.60, 33.58, 26.60, 21.19; HRMS (ESI, m/z) calculated for C₂₁H₂₆ClN₆O [M+H]⁺ 413.1851, found 413.1853.

### 4-((cis-14(6-Cyanopyridin-2-yl)methyl)-2-methylpiperidin-4-yl)amino)-N-methyl-1H-pyrrolo[2,3-b]pyridine-5-carboxamide (47b)

The title compound was synthesized according to General Procedure C. Yield: 55.0%; ¹H NMR (400 MHz, chloroform-d) δ 9.21 (d, J=7.9 Hz, 1H), 8.19 (s, 1H), 7.85-7.76 (m, 2H), 7.58 (dd, J=6.7, 2.0 Hz, 1H), 7.05 (d, J=3.6 Hz, 1H), 6.52 (d, J=3.7 Hz, 1H), 6.28 (s, 1H), 4.06-3.94 (m, 1H), 3.84 (dd, J=276.3, 15.3 Hz, 2H), 2.98 (d, J=4.7 Hz, 3H), 2.88-2.81 (m, 1H), 2.65-2.51 (m, 1H), 2.42-2.28 (m, 1H), 2.23-2.11 (m, 2H), 1.70-1.56 (m, 1H), 1.56-1.44 (m, 1H), 1.17 (d, J=6.1 Hz, 3H); ¹³C NMR (100 MHz, chloroform-d) δ 170.68, 162.72, 149.95, 148.45, 142.70, 137.57, 132.62, 126.97, 126.65, 121.50, 117.26, 105.30, 103.62, 102.62, 58.88, 56.16, 52.55, 50.97, 42.15, 33.42, 26.33, 21.04; HRMS (ESI, m/z) calculated for C₂₂H₂₆N₇O [M+H]⁺ 404.2193, found 404.2200.

### 4-((cis-14(5-Cyanopyridin-2-yl)methyl)-2-methylpiperidin-4-yl)amino)-N-methyl-1H-pyrrolo[2,3-b]pyridine-5-carboxamide (47c)

The title compound was synthesized according to General Procedure C. Yield: 74.0%; ¹H NMR (400 MHz, chloroform-d) δ 9.19 (d, J=7.9 Hz, 1H), 8.81 (d, J=1.4 Hz, 1H), 8.22 (s, 1H), 7.94 (dd, J=8.2, 2.1 Hz, 1H), 7.70 (d, J=8.2 Hz, 1H), 7.05 (d, J=3.6 Hz, 1H), 6.52 (d, J=3.7 Hz, 1H), 6.29 (s, 1H), 4.07-3.92 (m, 1H), 3.86 (dd, J=282.7, 15.7 Hz, 2H), 2.98 (d, J=4.7 Hz, 3H), 2.91-2.83 (m, 1H), 2.61-2.50 (m, 1H), 2.42-2.31 (m, 1H), 2.24-2.11 (m, 2H), 1.71-1.60 (m, 1H), 1.52 (q, J=11.5 Hz, 1H), 1.16 (d, J=6.1 Hz, 3H); ¹³C NMR (100 MHz, chloroform-d) δ 170.7, 165.7, 151.9, 150.1, 149.7, 142.9, 139.6, 122.7, 121.4, 117.0, 108.1, 105.4, 103.7, 102.8, 59.7, 56.4, 53.0, 51.2, 42.4, 33.7, 26.7, 21.4; HRMS (ESI, m/z) calculated for C₂₂H₂₆N₇O [M+H]⁺ 404.2193, found 404.2191.

### N-Methyl-4-((cis-2-methyl-14(5-(trifluoromethyl)pyridin-2-yl)methyl)piperidin-4-yl)amino)-1H-pyrrolo[2,3-b]pyridine-5-carboxamide (47d)

The title compound was synthesized according to General Procedure C. Yield: 32.5%; ¹H NMR (400 MHz, chloroform-d) δ 9.22 (d, J=7.8 Hz, 1H), 8.80 (s, 1H), 8.16 (s, 1H), 7.91 (dd, J=8.3, 2.0 Hz, 1H), 7.67 (d, J=8.2 Hz, 1H), 7.06 (d, J=3.6 Hz, 1H), 6.53 (d, J=3.7 Hz, 1H), 6.23-6.12 (m, 1H), 4.24 (d, J=15.2 Hz, 1H), 4.07-3.94 (m, 1H), 3.52 (d, J=15.2 Hz, 1H), 2.97 (d, J=4.7 Hz, 3H), 2.92-2.81 (m, 1H), 2.62-2.50 (m, 1H), 2.41-2.27 (m, 1H), 2.16 (dd, J=21.0, 13.9 Hz, 2H), 1.74-1.60 (m, 1H), 1.60-1.49 (m, 1H), 1.19 (d, J=6.1 Hz, 3H); ¹³C NMR (100 MHz, chloroform-d) δ 170.02, 164.86 (d, J_{C-F}=1.24 Hz), 149.70, 148.76, 146.63 (q, J_{C-F}=3.9 Hz), 141.69, 133.66 (q, J_{C-F}=3.3 Hz), 125.03 (q, J_{C-F}=33 Hz), 123.83 (q, J_{C-F}=274 Hz), 121.99, 120.95, 104.88, 103.12, 102.37, 58.95, 55.82, 52.27, 50.76, 41.86, 33.13, 26.20, 20.80; HRMS (ESI, m/z) calculated for C₂₂H₂₆F₃N₆O [M+H]⁺ 447.2115, found 447.2113.

### 4-((cis-14(6-Chloropyridin-3-yl)methyl)-2-methylpiperidin-4-yl)amino)-N-methyl-1H-pyrrolo[2,3-b]pyridine-5-carboxamide (48a)

The title compound was synthesized according to General Procedure C. Yield: 62.1%; ¹H NMR (400 MHz, chloroform-d) δ 9.16 (d, J=7.7 Hz, 1H), 8.30 (d, J=2.0 Hz, 1H), 8.22 (s, 1H), 7.68 (dd, J=8.2, 2.3 Hz, 1H), 7.30 (d, J=8.2 Hz, 1H), 7.03 (d, J=3.5 Hz, 1H), 6.52 (s, 1H), 6.48 (d, J=3.7 Hz, 1H), 4.01-3.85 (m, 1H), 3.61 (dd, J=365.0, 14.0 Hz, 2H), 2.96 (d, J=4.7 Hz, 3H), 2.81 (dd, J=8.8, 3.2 Hz, 1H), 2.44 (dd, J=8.4, 6.0 Hz, 1H), 2.18-2.05 (m, 3H), 1.62-1.44 (m, 2H), 1.22 (d, J=6.0 Hz, 3H); ¹³C NMR (100 MHz, chloroform-d) δ 170.59,150.03,149.96,149.76, 149.32, 142.96, 139.39, 133.94, 124.07, 121.28, 105.18, 103.62, 102.68, 56.05, 54.02, 51.59, 51.10, 42.22, 33.37 21.19; HRMS (ESI, m/z) calculated for C₂₁H₂₆ClN₆O [M+H]⁺ 413.1851, found 413.1853.

### 4-((cis-14(6-Cyanopyridin-3-yl)methyl)-2-methylpiperidin-4-yl)amino)-N-methyl-1H-pyrrolo[2,3-b]pyridine-5-carboxamide (48b)

The title compound was synthesized according to General Procedure C. Yield: 54.3%; ¹H NMR (400 MHz, chloroform-d) δ 9.13 (d, J=7.9 Hz, 1H), 8.66 (d, J=1.6 Hz, 1H), 8.24 (s, 1H), 7.86 (dd, J=8.0, 2.0 Hz, 1H), 7.71-7.62 (m, 1H), 7.07 (d, J=3.6 Hz, 1H), 6.51 (d, J=3.7 Hz, 1H), 6.28-6.13 (m, 1H), 4.05-3.91 (m, 1H), 3.70 (dd, J=365.2, 14.7 Hz, 2H), 2.98 (d, J=4.8 Hz, 3H), 2.83-2.73 (m, 1H), 2.54-2.45 (m, 1H), 2.24-2.16 (m, 2H), 2.16-2.07 (m, 1H), 1.64 1.44 (m, 2H), 1.19 (d, J=6.1 Hz, 3H); ¹³C NMR (100 MHz, chloroform-d) δ 170.85, 151.44, 150.44, 149.94, 143.60, 140.11, 137.03, 132.42, 128.33, 121.32, 117.51, 105.27, 103.71, 102.72, 56.37, 54.81, 52.24, 51.12, 42.39, 33.56 21.39; HRMS (ESI, m/z) calculated for C₂₂H₂₆N₇O [M+H]⁺ 404.2193, found 404.2197.

### N-Methyl-4-((cis-2-methyl-1-((6-(trifluoromethyl)pyridin-3-yl)methyl)piperidin-4-yl)amino)-1H-pyrrolo[2,3-b]pyridine-5-carboxamide (48c)

The title compound was synthesized according to General Procedure C. Yield: 48.4%; ¹H NMR (400 MHz, chloroform-d) δ 9.21 (d, J=7.8 Hz, 1H), 8.66 (s, 1H), 8.21 (s, 1H), 7.89 (d, J=8.0 Hz, 1H), 7.66 (d, J=8.0 Hz, 1H), 7.03 (d, J=3.4 Hz, 1H), 6.48 (d, J=3.6 Hz, 1H), 6.45 (s, 1H), 4.04-3.87 (m, 1H), 3.71 (dd, J=364.9, 14.4 Hz, 2H), 2.96 (d, J=4.6 Hz, 3H), 2.81 (d, J=11.7 Hz, 1H), 2.48 (dd, J=8.4, 6.0 Hz, 1H), 2.14 (dd, J=25.0, 12.5 Hz, 3H), 1.64-1.44 (m, 2H), 1.22 (d, J=6.0 Hz, 3H); ¹³C NMR (100 MHz, chloroform-d) δ 170.62, 150.27, 150.16, 149.31, 147.02 (q, J_{C-F}=34.6 Hz), 142.54, 138.87, 137.60, 121.79 (q, J_{C-F}=273.8 Hz), 121.51, 120.32 (q, J_{C-F}=2.6 Hz), 105.37, 103.68, 102.77, 56.27, 54.64, 51.99, 51.22, 42.32, 33.49, 26.73, 21.36; HRMS (ESI, m/z) calculated for C₂₂H₂₆F₃N₆O [M+H]⁺ 447.2115, found 447.2114.

### N-Methyl-4-((cis-2-methyl-1-((2-(trifluoromethyppyrimidin-5-yl)methyl)piperidin-4-yl)amino)-1H-pyrrolo[2,3-b]pyridine-5-carboxamide (49)

The title compound was synthesized according to General Procedure C. Yield: 33.0%; ¹H NMR (400 MHz, chloroform-d) δ 9.16 (d, J=7.7 Hz, 1H), 8.87 (s, 2H), 8.21 (s, 1H), 7.07 (d, J=3.4 Hz, 1H), 6.51 (d, J=3.5 Hz, 1H), 6.15 (s, 1H), 4.06-3.94 (m, 1H), 3.74 (dd, J=352.7, 14.8 Hz, 2H), 2.98 (d, J=4.6 Hz, 3H), 2.82 (d, J=11.8 Hz, 1H), 2.52 (s, 1H), 2.27-2.09 (m, 3H), 1.67-1.48 (m, 2H), 1.23 (d, J=6.0 Hz, 3H); ¹³C NMR (100 MHz, chloroform-d) 6 170.77, 158.06, 155.69 (q, J_{C-F}=36 Hz), 150.24, 149.99, 143.39, 135.63, 119.68 (q, J_{C-F}=276 Hz), 105.30, 103.78, 102.79, 56.37, 52.44, 52.20, 51.09, 42.27, 33.48, 26.75, 21.39; HRMS (ESI, m/z) calculated for C₂₁H₂₅F₃N₇O [M+H]⁺ 448.2067, found 448.2077.

### General Procedure D

DIEA and 4-chloro-N-methyl-1H-pyrrolo[2,3-b]pyridine-5-carboxamide (0.5 mmol) were added to a solution of the amine intermediates 40-42 (4 eq) in NMP (2 mL). The mixture was stirred at 180 ° C. for at least 12 h. The resulting mixture was cooled to room temperature, diluted with ethyl acetate, washed with H₂O and brine, dried over MgSO₄, and purified by silica gel flash chromatography (dichloromethane:methanol=10:1 elution) to afford the title products 50a-c.

### 4-((1-Benzyl-2,2-dimethylpiperidin-4-yl)amino)-N-methyl-1H-pyrrolo[2,3-b]pyridine-5-carboxamide (50a)

The title compound was synthesized according to General Procedure D. Yield: 62.3%; ¹H NMR (400 MHz, chloroform-d) δ 9.14 (d, J=7.8 Hz, 1H), 8.25 (s, 1H), 7.37 (d, J=7.1 Hz, 2H), 7.31 (t, J=7.4 Hz, 2H), 7.23 (t, J=7.2 Hz, 1H), 6.99 (d, J=3.1 Hz, 1H), 6.56 (s, 1H), 6.53 (d, J=3.3 Hz, 1H), 4.19-4.09 (m, 1H), 3.56 (dd, J=397.7, 14.1 Hz, 2H), 2.98 (d, J=4.7 Hz, 3H), 2.72-2.61 (m, 1H), 2.47-2.34 (m, 1H), 2.11-1.97 (m, 2H), 1.67 (t, J=12.1 Hz, 1H), 1.61-1.49 (m, 1H), 1.27 (s, 3H), 1.18 (s, 3H); ¹³C NMR (101 MHz, chloroform-d) δ 170.47, 150.11, 142.83, 142.73, 141.05, 128.23, 128.18, 126.60, 121.03, 105.17, 103.68, 102.79, 54.17, 53.32, 48.40, 47.37, 45.53, 34.25, 30. 26.58, 16.51; HRMS (ESI, m/z) calculated for C₂₃H₃ON₅O [M+H]⁺ 392.2445, found 392.2447.

### 4-((1-(3-Chlorobenzyl)-2,2-dimethylpiperidin-4-yl)amino)-N-methyl-1H-pyrrolo[2,3-b]pyridine-5-carboxamide (50b)

The title compound was synthesized according to General Procedure D. Yield: 11.0%; ¹H NMR (400 MHz, chloroform-d) δ 9.10 (d, J=7.8 Hz, 1H), 8.23 (s, 1H), 7.38 (s, 1H), 7.24-7.14 (m, 3H), 7.04 (d, J=3.5 Hz, 1H), 6.57 (d, J=3.5 Hz, 1H), 6.21 (s, 1H), 4.26-4.09 (m, 1H), 3.54 (dd, J=384.1, 14.3 Hz, 2H), 2.98 (d, J=4.7 Hz, 3H), 2.67-2.58 (m, 1H), 2.48-2.40 (m, 1H), 2.12-2.05 (m, 1H), 2.05-1.99 (m, 1H), 1.67 (t, J=12.1 Hz, 1H), 1.63-1.50 (m, 1H), 1.25 (s, 3H), 1.18 (s, 3H); ¹³C NMR (101 MHz, chloroform-d) δ 170 .63, 149.97, 143.44, 143.39, 143.35, 134.18, 129.41, 128.15, 126.77, 126.26, 120.93, 105.18, 103.70, 102.83, 54.22, 52.94, 48.28, 47.37, 45.75, 34.25, 30.37, 26.59, 16.63; HRMS (ESI, m/z) calculated for C₂₃H₂₉ClN₅O [M+H]⁺ 426.2055, found 426.2059.

### 4-((1-(4-Chlorobenzyl)-2,2-dimethylpiperidin-4-ypamino)-N-methyl-1H-pyrrolo[2,3-b]pyridine-5-carboxamide (50c)

The title compound was synthesized according to General Procedure D. Yield: 10.4%; ¹H NMR (400 MHz, Methanol-d4) δ 8.36 (s, 1H), 7.63-7.50 (m, 4H), 7.40 (d, J=3.5 Hz, 1H), 6.96 (d, J=3.6 Hz, 1H), 4.68-4.58 (m, 1H), 4.40 (dd, J=318.5, 13.2 Hz, 2H), 3.76-3.58 (m, 1H), 3.59-3.50 (m, 1H), 3.43 (d, J=10.4 Hz, 1H), 2.88 (s, 3H), 2.42 (d, J=11.6 Hz, 1H), 2.09 (t, J=13.0 Hz, 1H), 1.89-1.78 (m, 1H), 1.75 (s, 3H), 1.73 (s, 3H); ¹³C NMR (100 MHz, Methanol-d4) δ 169.19, 139.72, 137.46, 135.85, 134.49, 134.35, 130.55, 129.57, 125.39, 106.45, 105.21, 104.15, 65.25, 54.36, 47.40, 47.36, 44.51, 31.19, 27.29, 26.74, 18.54; HRMS (ESI, m/z) calculated for C₂₃H₂₉ClN₅O [M+H]⁺ 426.2055, found 426.2058.

### General Procedure E

Acetic acid and triacetoxyborohydride (4.7 mmol) were added to the pure enantiomers N-Boc-2-methyl-4-piperidone (51a,b; 2.35 mmol) and a solution of stereospecific 1-phenylethan-1-amine (4.7 mmol) in DCM (10 mL). The mixture was stirred at room temperature for at least 12 h. The resulting mixture was added dropwise with a saturated aqueous NaHCO₃ solution, diluted with ethyl acetate, washed with H₂O and brine, and dried over MgSO₄. The resulting organic layer was purified by silica gel flash chromatography (hexane:ethyl acetate=4:1 elution) to afford the title products 53a and 53b.

### tent-Butyl (2S,4S)-2-methyl-4-(((S)-1-phenylethyl)amino)piperidine-1-carboxylate (53a)

The title compound was synthesized according to General Procedure E. Yield: 88.2%; ¹H NMR (400 MHz, chloroform-d) δ 7.34-7.25 (m, 4H), 7.25-7.19 (m, 1H), 4.03 (h, J=6.6 Hz, 1H), 3.83 (q, J=6.6 Hz, 1H), 3.64 (ddd, J=13.5, 5.5, 3.0 Hz, 1H), 3.18 (ddd, J=13.5, 11.9, 4.0 Hz, 1H), 2.73 (dt, J=9.3, 4.7 Hz, 1H), 1.77 (ddd, J=13.9, 6.3, 3.9 Hz, 1H), 1.68 (ddt, J=13.4, 11.0, 5.4 Hz, 1H), 1.53-1.45 (m, 1H), 1.43 (s, 9H), 1.34 (d, J=1.4 Hz, 3H), 1.32 (d, J=1.6 Hz, 3H), 1.30-1.25 (m, 1H); ¹³C NMR (100 MHz, chloroform-d) δ 155.0, 146.1, 128.5, 126.8, 126.4, 79.0, 55.5, 48.4, 46.9, 35.1, 34.2, 31.4, 28.5, 24.9, 19.6; MS (ESI, m/z) calculated for C₁₉H₃₁N₂O₂ [M+H]⁺ 319.24, found 319.15.

### tert-Butyl (2R,4R)-2-methyl-4-(((R)-1-phenylethyl)amino)piperidine-1-carboxylate (53b)

The title compound was synthesized according to General Procedure E. Yield: 86.1%; ¹H NMR (400 MHz, chloroform-d) δ 7.35-7.18 (m, 5H), 4.03 (h, J=6.6 Hz, 1H), 3.83 (q, J=6.6 Hz, 1H), 3.64 (ddd, J=13.5, 5.5, 3.0 Hz, 1H), 3.18 (ddd, J=13.5, 11.9, 4.0 Hz, 1H), 2.73 (dt, J=9.4, 4.7 Hz, 1H), 1.77 (ddd, J=13.9, 6.3, 3.9 Hz, 1H), 1.68 (ddt, J=13.4, 11.1, 5.4 Hz, 1H), 1.51-1.45 (m, 1H), 1.43 (s, 9H), 1.34 (d, J=1.5 Hz, 3H), 1.32 (d, J=1.6 Hz, 3H), 1.31-1.26 (m, 1H); ¹³C NMR (100 MHz, chloroform-d) δ 155.0, 146.1, 128.5, 126.8, 126.3, 79.0, 55.5, 48.4, 46.9, 35.1, 34.2, 31.3, 28.5, 24.9, 19.5; MS (ESI, m/z) calculated for C₁₉H₃₁N₂O₂ [M+H]⁺ 319.24, found 319.15.

### General Procedure F

Acetic acid (1 eq) was slowly added to 53a and 53b (1 eq) and a solution of 10% palladium hydroxide (20 wt. % on carbon) in methanol. The mixture was subjected to hydrogenation using a 5-layer balloon filled with hydrogen at 50° C. for 2 h. The reaction mixture was filtered through Celite and washed with methanol. The solvent was removed from the combined filtrate using a rotary evaporator to afford the products 54a-b as white solids.

### tent-Butyl (2S,4S)-4-amino-2-methylpiperidine-1-carboxylate (54a)

The title compound was synthesized according to General Procedure F. Yield: 92.9%; ¹H NMR (400 MHz, Methanol-d4) 6 3.96-3.86 (m, 1H), 3.69 (ddd, J=14.2, 6.6, 3.9 Hz, 1H), 3.39-3.32 (m, 2H), 2.16 (ddt, J=19.6, 11.3, 4.4 Hz, 1H), 2.06 (dt, J=13.3, 5.0 Hz, 1H), 1.66-1.58 (m, 1H), 1.58-1.48 (m, 1H), 1.46 (s, 9H), 1.27 (d, J=6.6 Hz, 3H); ¹³C NMR (100 MHz, Methanol-d4) δ 155.2, 80.0, 48.4, 45.3, 36.4, 33.3, 27.6, 27.3, 18.5; MS (ESI, m/z) calculated for C₁₁H₂₃N₂O₂ [M+H]⁺ 215.18, found 215.20; Chirality was confirmed using (S)-Mosher's reagent, 29a-ms; ¹H NMR (400 MHz, chloroform-d) δ 7.51 (dd, J=6.4, 2.6 Hz, 2H), 7.41 (dt, J=4.6, 2.8 Hz, 3H), 6.95 (d, J=7.2 Hz, 1H), 4.23-4.09 (m, 2H), 3.84 (ddd, J=14.1, 5.3, 3.2 Hz, 1H), 3.47-3.36 (m, 3H), 3.07 (ddd, J=14.2, 11.9, 3.8 Hz, 1H), 2.00-1.85 (m, 2H), 1.73-1.65 (m, 1H), 1.61-1.54 (m, 1H), 1.45 (s, 9H), 1.15 (d, J=7.0 Hz, 3H); ¹³C NMR (101 MHz, chloroform-d) δ 165.6, 154.9, 132.4, 129.7, 128.8, 127.8 (d, J_{C-F}=1.36 Hz), 123.9 (q, J_{C-F}=290.0 Hz), 84.1 (q, J_{C-F}=26.3 Hz), 79.8, 55.1, 46.3, 43.6, 35.1, 34.4, 29.6, 28.6, 19.0; 19 F NMR (377 MHz, chloroform-d) δ-68.8023; MS (ESI, m/z) calculated for C₂₁H₃₀F₃N₂O₄ [M+H]⁺ 431.22, found 431.15.

### tent-Butyl (2R,4R)-4-amino-2-methylpiperidine-1-carboxylate (54b)

The title compound was synthesized according to General Procedure F. Yield: 96.1%; ¹H NMR (400 MHz, Methanol-d4) δ 3.96-3.84 (m, 1H), 3.69 (ddd, J=14.2, 6.6, 3.9 Hz, 1H), 3.39-3.32 (m, 2H), 2.17 (ddt, J=13.9, 9.1, 6.9 Hz, 1H), 2.07 (dt, J=13.2, 4.7 Hz, 1H), 1.69-1.52 (m, 2H), 1.46 (s, 9H), 1.28 (d, J=6.6 Hz, 3H); ¹³C NMR (100 MHz, Methanol-d4) δ 155.1, 80.0, 48.4, 45.4, 36.5, 33.2, 27.5, 27.3, 18.5; MS (ESI, m/z) calculated for C₁₁H₂₃N₂O₂ [M+H]⁺ 215.18, found 215.15; Chirality was confirmed using (S)-Mosher's reagent, 29b-ms; ¹H NMR (400 MHz, chloroform-d) δ 7.50 (dd, J=6.4, 2.6 Hz, 2H), 7.41 (dt, J=4.5, 2.8 Hz, 3H), 7.01 (d, J=7.1 Hz, 1H), 4.25-4.16 (m, 1H), 4.13 (ddd, J=10.7, 7.4, 5.3 Hz, 1H), 3.83 (ddd, J=14.0, 5.5, 3.2 Hz, 1H), 3.41-3.35 (m, 3H), 3.04 (ddd, J=14.2, 11.9, 3.8 Hz, 1H), 2.03-1.94 (m, 1H), 1.90 (ddd, J=11.9, 8.3, 5.7 Hz, 1H), 1.69-1.63 (m, 2H), 1.46 (s, 9H), 1.22 (d, J=7.0 Hz, 3H); ¹³C NMR (100 MHz, chloroform-d) δ 165.6, 154.8, 132.3, 129.6, 128.7, 127.7 (d, J_{C-F}=1.39 Hz), 123.9 (q, J_{C-F}=289.9 Hz), 84.0 (q, J_{C-F}=26.4 Hz), 79.7, 55.0, 46.4, 43.5, 35.0, 34.4, 29.4, 28.5, 18.9; 19 F NMR (377 MHz, chloroform-d) δ-68.6802; MS (ESI, m/z) calculated for C₂₁H₃₀F₃N₂O₄ [M+H]⁺ 431.22, found 431.15.

### N-Methyl-4-(((2S,4S)-2-methylpiperidin-4-yl)amino)-1H-pyrrolo[2,3-b]pyridine-5-carboxamide hydrochloride (55a)

The title compound was synthesized according to General Procedure A. Yield: 25.9%; ¹H NMR (400 MHz, Deuterium Oxide) δ 8.19 (s, 1H), 7.33 (d, J=3.7 Hz, 1H), 6.82 (d, J=3.8 Hz, 1H), 4.53-4.42 (m, 1H), 3.65-3.46 (m, 3H), 3.27 (td, J=13.4, 2.7 Hz, 1H), 2.90 (s, 3H), 2.53-2.35 (m, 2H), 1.82 (qd, J=13.9, 4.3 Hz, 1H), 1.75-1.60 (m, 1H), 1.39 (d, J=6.5 Hz, 3H); ¹³C NMR (100 MHz, Deuterium Oxide) δ 169.4, 151.7, 138.8, 134.7, 124.5, 106.9, 106.7, 104.4, 52.3, 49.4, 43.2, 37.1, 29.0, 26.9, 18.9; MS (ESI, m/z) calculated for C₁₅H₂₂N₅O [M+H]⁺ 288.18, found 288.15.

### N-Methyl-4-(((2R,4R)-2-methylpiperidin-4-yl)amino)-1H-pyrrolo[2,3-b]pyridine-5-carboxamide hydrochloride (55b)

The title compound was synthesized according to General Procedure A. Yield: 61.5%; ¹H NMR (400 MHz, Deuterium Oxide) δ 8.11 (s, 1H), 7.29 (d, J=3.6 Hz, 1H), 6.71 (d, J=3.7 Hz, 1H), 4.39-4.25 (m, 1H), 3.65-3.48 (m, 2H), 3.27 (td, J=13.3, 2.6 Hz, 1H), 2.90 (s, 3H), 2.48-2.32 (m, 2H), 1.78 (qd, J=14.0, 4.2 Hz, 1H), 1.64 (q, J=12.1 Hz, 1H), 1.41 (d, J=6.5 Hz, 3H); ¹³C NMR (100 MHz, Deuterium Oxide) δ 169.0, 150.6, 139.7, 135.5, 123.7, 106.0, 105.3, 103.4, 51.7, 48.6, 42.6, 36.7, 28.5, 26.2, 18.3; MS (ESI, m/z) calculated for C₁₅H₂₂N₅O [M+H]⁺ 288.18, found 288.15.

### 4-(42S,4S)-1-(4-Chlorobenzyl)-2-methylpiperidin-4-yl)amino)-N-methyl-1H-pyrrolo[2,3-b]pyridine-5-carboxamide (56a)

The title compound was synthesized according to General Procedure C. Yield: 28.5%; ¹H NMR (400 MHz, chloroform-d) δ 9.13 (d, J=7.8 Hz, 1H), 8.21 (s, 1H), 7.31-7.23 (m, 4H), 7.02 (d, J=3.4 Hz, 1H), 6.48 (d, J=3.6 Hz, 1H), 6.33 (s, 1H), 4.07 (d, J=13.6 Hz, 1H), 3.92 (dq, J=11.4, 7.7, 5.6 Hz, 1H), 3.12 (d, J=13.6 Hz, 1H), 2.96 (d, J=4.6 Hz, 3H), 2.90-2.77 (m, 1H), 2.42 (ddd, J=10.7, 6.0, 2.2 Hz, 1H), 2.15 (d, J=12.2 Hz, 1H), 2.11-2.02 (m, 2H), 1.63-1.44 (m, 2H), 1.22 (d, J=6.0 Hz, 3H); ¹³C NMR (100 MHz, chloroform-d) δ 170.7, 150.1, 149.7, 143.0, 138.0, 132.6, 130.3, 128.5, 121.3, 105.3, 103.7, 102.9, 57.0, 56.1, 51.7, 51.4, 42.4, 33.6, 26.7, 21.3; HRMS (ESI, m/z) calculated for C₂₂H₂₇ClN₅O [M+H]⁺ 412.1899, found 412.1902; enantiomeric purity: 99.9% ee.

### 4-(42R,4R)-1-(4-Chlorobenzyl)-2-methylpiperidin-4-yl)amino)-N-methyl-1H-pyrrolo[2,3-b]pyridine-5-carboxamide (56b)

The title compound was synthesized according to General Procedure C. Yield: 25.9%; ¹H NMR (400 MHz, chloroform-d) δ 10.50 (s, 1H), 9.08 (d, J=7.9 Hz, 1H), 8.21 (s, 1H), 7.33-7.24 (m, 4H), 7.05 (d, J=3.6 Hz, 1H), 6.53 (d, J=3.7 Hz, 1H), 6.17-6.00 (m, 1H), 3.94 (dp, J=11.5, 3.8 Hz, 1H), 3.60 (dd, J=382.4, 13.6 Hz, 2H), 2.97 (d, J=4.8 Hz, 3H), 2.90-2.78 (m, 1H), 2.43 (ddd, J=11.0, 6.1, 2.5 Hz, 2H), 2.20-2.13 (m, 1H), 2.13-2.04 (m, 2H), 1.63-1.45 (m, 2H), 1.23 (d, J=6.1 Hz, 3H); ¹³C NMR (100 MHz, chloroform-d) δ 170.9, 150.6, 150.0, 143.8, 138.1, 132.6, 130.3, 128.5, 121.1, 105.2, 103.0, 57.0, 56.1, 51.7, 51.4, 42.5, 33.6, 26.7, 21.4; HRMS (ESI, m/z) calculated for C₂₂H₂₇ClN₅O [M+H]⁺ 412.1899, found 412.1904; enantiomeric purity: 99.6% ee.

### 4-(42S,4S)-1-(4-trifluoromethyl)-2-methylpiperidin-4-yl)amino)-N-methyl-1H-pyrrolo[2,3-b]pyridine-5-carboxamide (57a)

The title compound was synthesized according to General Procedure C. Yield: 51%; ¹H NMR (400 MHz, chloroform-d) δ 9.15 (d, J=7.9 Hz, 1H), 8.21 (s, 1H), 7.57 (d, J=8.1 Hz, 2H), 7.46 (d, J=8.0 Hz, 2H), 7.03 (d, J=3.6 Hz, 1H), 6.50 (d, J=3.7 Hz, 1H), 6.27 (s, 1H), 4.16 (d, J=14.0 Hz, 1H), 3.94 (ddd, J=11.4, 7.6, 3.9 Hz, 1H), 3.19 (d, J=14.0 Hz, 1H), 2.97 (d, J=4.7 Hz, 3H), 2.90-2.76 (m, 1H), 2.46 (ddd, J=10.9, 6.1, 2.4 Hz, 1H), 2.23-2.04 (m, 3H), 1.66-1.45 (m, 2H), 1.22 (d, J=6.1 Hz, 3H); ¹³C NMR (100 MHz, chloroform-d) δ 170.74, 150.09, 149.85, 144.09, 143.21, 129.25 (q, J_{C-F}=32.2 Hz), 129.02, 125.29 (q, J_{C-F}=3.7 Hz), 124.44 (q, J_{C-F}=271.9 Hz), 121.24, 105.29, 103.72, 102.90, 57.32, 56.26, 51.96, 51.35, 42.48, 33.62, 26.74, 21.38; HRMS (ESI, m/z) calculated for C₂₃H₂₇F₃N₅O [M+H]⁺ 446.2162, found 446.2166; enantiomeric purity: 99.9% ee.

### 4-(42R,4R)-1-(4-trifluoromethyl)-2-methylpiperidin-4-yl)amino)-N-methyl-1H-pyrrolo[2,3-b]pyridine-5-carboxamide (57b)

The title compound was synthesized according to General Procedure C. Yield: 34%; ¹H NMR (400 MHz, chloroform-d) δ 9.15 (d, J=7.9 Hz, 1H), 8.21 (s, 1H), 7.57 (d, J=8.1 Hz, 2H), 7.46 (d, J=8.0 Hz, 2H), 7.03 (d, J=3.6 Hz, 1H), 6.50 (d, J=3.7 Hz, 1H), 6.27 (s, 1H), 4.16 (d, J=14.0 Hz, 1H), 3.94 (ddd, J=11.4, 7.6, 3.9 Hz, 1H), 3.19 (d, J=14.0 Hz, 1H), 2.97 (d, J=4.7 Hz, 3H), 2.90-2.76 (m, 1H), 2.46 (ddd, J=10.9, 6.1, 2.4 Hz, 1H), 2.23-2.04 (m, 3H), 1.66-1.45 (m, 2H), 1.22 (d, J=6.1 Hz, 3H); ¹³C NMR (100 MHz, chloroform-d) δ 170.75, 150.09, 149.84, 144.08, 143.21, 129.24 (q, J_{C-F}=32.2 Hz), 129.02, 125.29 (q, J_{C-F}=3.7 Hz), 124.43 (q, J_{C-F}=271.9 Hz), 121.28, 105.29, 103.69, 102.86, 57.18, 56.12, 51.82, 51.21, 42.34, 33.48, 26.60, 21.25; HRMS (ESI, m/z) calculated for C₂₃H₂₇F₃N₅O [M+H]⁺ 446.2162, found 446.2165; enantiomeric purity: absolute stereochemistry shown.

### 4-(428,48)-1-((6-Cyanopyridin-3-yl)methyl)-2-methylpiperidin-4-yl)amino)-N-methyl-1H-pyrrolo[2,3-b]pyridine-5-carboxamide (58a)

The title compound was synthesized according to General Procedure C. Yield: 76%; ¹H NMR (400 MHz, chloroform-d) δ 10.86 (s, 1H), 9.12 (d, J=7.9 Hz, 1H), 8.71-8.56 (m, 1H), 8.24 (s, 1H), 7.86 (dd, J=8.0, 1.9 Hz, 1H), 7.67 (d, J=7.9 Hz, 1H), 7.07 (d, J=3.6 Hz, 1H), 6.52 (d, J=3.7 Hz, 1H), 6.23-6.10 (m, 1H), 4.16 (d, J=14.7 Hz, 1H), 4.04-3.90 (m, 1H), 3.25 (d, J=14.7 Hz, 1H), 2.98 (d, J=4.7 Hz, 3H), 2.83-2.74 (m, 1H), 2.54-2.44 (m, 1H), 2.23-2.15 (m, 2H), 2.12 (d, J=12.5 Hz, 1H), 1.65 1.53 (m, 1H), 1.53-1.46 (m, 1H), 1.20 (d, J=6.1 Hz, 3H); ¹³C NMR (100 MHz, chloroform-d) δ 170.75, 151.32, 150.44, 149.80, 143.54, 139.99, 136.89, 132.31, 128.21, 121.17, 117.39, 105.14, 103.58, 102.60, 56.25, 54.69, 52.13, 51.00, 42.28, 33.45, 26.62, 21.27; HRMS (ESI, m/z) calculated for C₂₂H₂₅N₇O [M+H]⁺ 404.2193, found 404.2194; enantiomeric purity: 99.5% ee.

### 4-(42R,4R)-1-((6-Cyanopyridin-3-yl)methyl)-2-methylpiperidin-4-yl)amino)-N-methyl-1H-pyrrolo[2,3-b]pyridine-5-carboxamide (58b)

The title compound was synthesized according to General Procedure C. Yield: 39%; ¹H NMR (400 MHz, chloroform-d) δ 9.23 (d, J=7.8 Hz, 1H), 8.67 (d, J=1.6 Hz, 1H), 8.13 (s, 1H), 7.86 (dd, J=8.0, 2.0 Hz, 1H), 7.67 (d, J=8.0 Hz, 1H), 7.05 (d, J=3.6 Hz, 1H), 6.51 (d, J=3.7 Hz, 1H), 6.04 (s, 1H), 4.17 (d, J=14.7 Hz, 1H), 3.99 (dtd, J=11.5, 7.6, 4.0 Hz, 1H), 3.26 (d, J=14.7 Hz, 1H), 2.98 (d, J=4.8 Hz, 3H), 2.80 (dt, J=11.8, 3.3 Hz, 1H), 2.51 (ddd, J=10.9, 6.2, 2.5 Hz, 1H), 2.27-2.17 (m, 2H), 2.15-1.98 (m, 1H), 1.66-1.55 (m, 1H), 1.55-1.44 (m, 1H), 1.21 (d, J=6.1 Hz, 3H); ¹³C NMR (100 MHz, chloroform-d) δ 170.73, 151.32, 150.32, 149.82, 143.48, 139.99, 136.90, 132.30, 128.21, 121.20, 117.39, 105.15, 103.58, 102.60, 56.25, 54.69, 52.12, 51.00, 42.27, 33.44, 26.62, 21.27; HRMS (ESI, m/z) calculated for C₂₂H₂₅N₇O [M+H]⁺ 404.2193, found 404.2196; enantiomeric purity: absolute stereochemistry shown.

### Determination of Effects on Fibrosis Indications

A recent study on the effect of TGF-β on pro-fibrosis reveals that signal transducer and activator of transcription 3 (STAT3) is associated with fibrotic diseases.

STAT3 signal transduction is overactivated in systemic sclerosis in a transforming growth factor-β (TGF-β)-dependent manner. This signal suppression might ameliorate skin fibrosis in experimental mouse models.

It has also been reported that the JAK1/STAT3 pathway is also directly associated with TGF-β-induced liver fibrosis, an abnormal response to hepatic injury.

Thus, considering that JAK1 inhibitors may be potential candidates for the treatment of fibrotic diseases, a racemic mixture of 56a and 56b was selected as a representative compound 31g and its biological effects were investigated in liver fibrosis models.

### Effect on Human LX-2 Cell Proliferation

Hepatic stellate cells (HSCs), hepatocytes, and macrophages are main cell types of liver fibrosis. Resting HSCs are activated to transform into myofibroblast-like cells in response to chronic injury.

Accordingly, LX-2 human hepatic stellate cells were selected to determine their effect on liver fibrosis. To this end, HSC activation through proliferation and fibrogenic gene expression and migration was investigated.

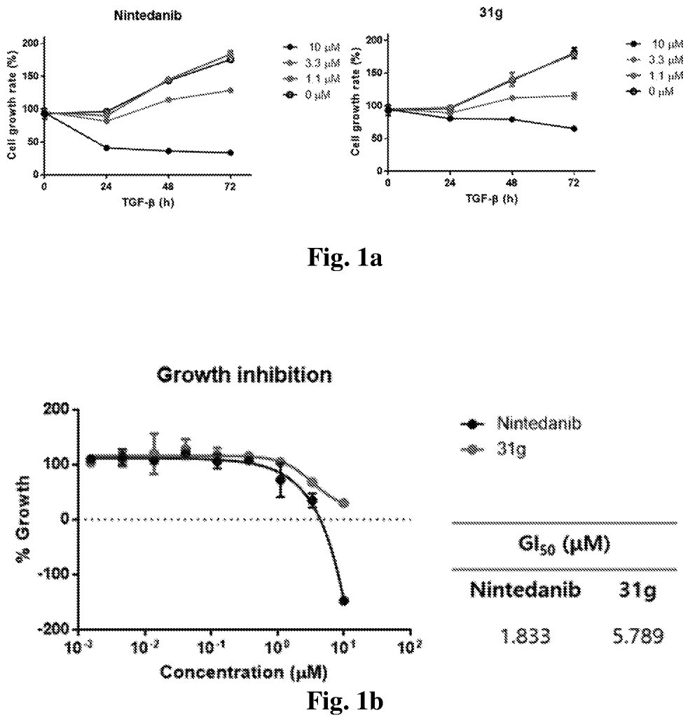

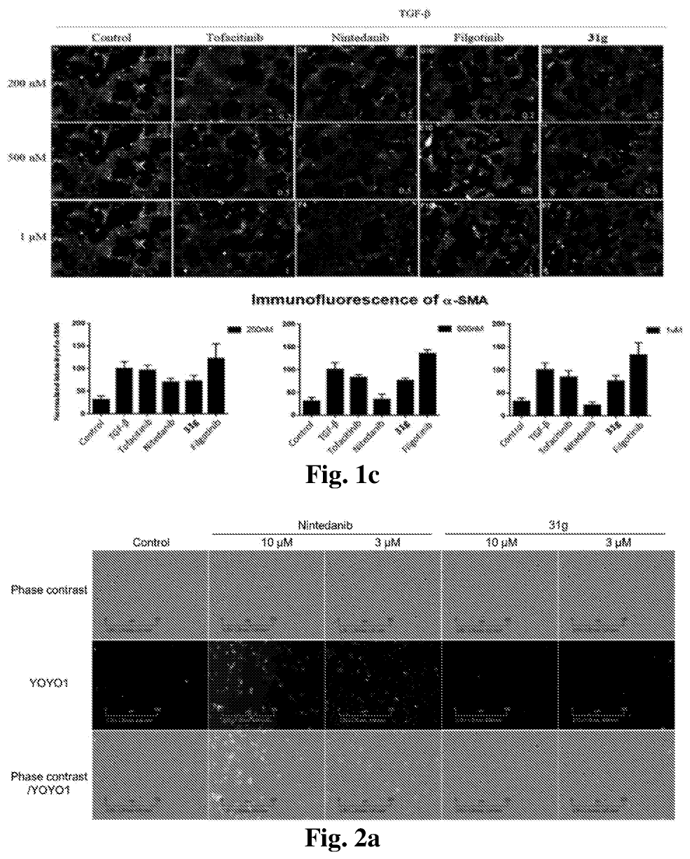

As a result of the testing, nintedanib, an FDA-approved drug for liver fibrosis, and the inventive compound showed similar inhibitory activities on LX-2 cell proliferation induced by TGF-β (see FIGS. 1a and 1b). That is, time-dependent inhibitory effects of nintedanib and 31g on LX-2 proliferation were observed, and nintedanib and 31g showed dose-dependent inhibitory effects on LX-2 proliferation under the same conditions.

After incubation of nintedanib or 31g with TGF-β for 72 h, its inhibitory effect on LX-2 cell growth was superior to those of tofacitinib or filgotinib (see FIG. 1c).

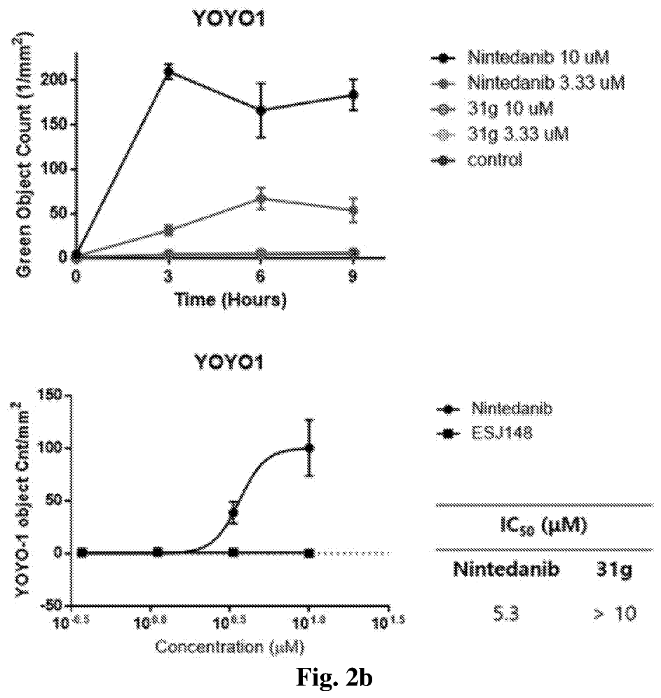

Furthermore, the potential toxicity of 31g was determined. To this end, the cytotoxicity of 31g was evaluated using a cell-impermeant cyanine dimer nucleic acid stain (YOYO1) that binds to dsDNA in human astrocytes. 31g had a low toxicity to LX-2 cells compared to nintedanib in general medium (see FIGS. 2a and 2b).

For reference, YOYO1 images were analyzed in a time-dependent manner by INcucyte Imaging FLR and IC₅₀ values were calculated by the imaging FLR.

### Effect of Inhibiting TGF-β-Induced Fibrosis Gene Expression and Migration in Human LX-2 Cells

TGF-β is a potent profibrotic cytokine that regulates genes involved in liver fibrosis and 11 TGF-β-induced HSCs differentiate into myofibroblasts and secrete extracellular matrix (ECM) proteins. Thus, changes in fibrosis genetic parameters were measured to investigate the effect of 31g on myoblast differentiation in HSC activation.

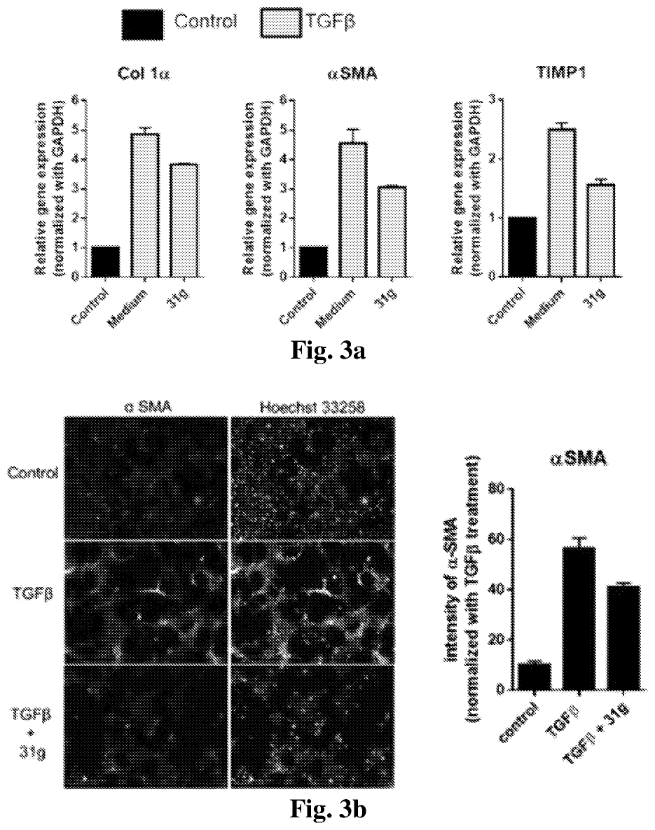

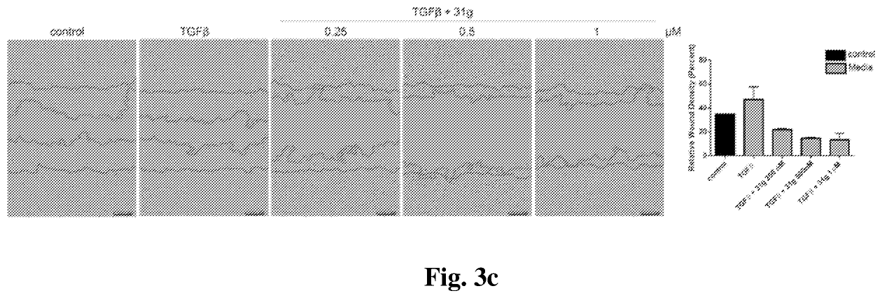

As shown in FIG. 3a, TGF-β-induced HSCs produced tissue inhibitors of a-smooth muscle actin (α-SMA), type 1 collagen α1 (CollA1), and metalloproteinase 1 (TIMP1), and treatment with 31g at a concentration of 500 nM decreased the production of α-SMA, Co11A1, and TIMP1 in HSCs.

Particularly, immunofluorescence staining and intensity analyses using Harmony 3.1 software revealed that treatment with 31g (250 nM) decreased the production of α-SMA in LX-2 cells (see FIG. 3b).

Since HSCs migrate to the sites of tissue injury during fibrogenesis in liver fibrosis and differentiate into contractile myofibroblasts that promote liver stiffness, the migration of HSCs was examined using wound-healing assays. As a result, 250 nM 31g was found to significantly inhibit TGF-β-induced migration of human HSCs (see FIG. 3c). In order to exclude the proliferation effect, wound healing was observed in a fusion and serum-deprived state. There were no differences even during proliferation at concentrations of 0.25 μM, 0.5 μM, and 1 μM, where migration-inhibitory effects were observed.

31g inhibited the migration of HSCs more effectively than tofacitinib or filgotinib when each drug (500 nM) was incubated with TGF-β (10 ng/mL) (FIG. 1c).

These results demonstrated the potential of 31g as a therapeutic compound to prevent HSC activation.

### Effect of Reducing JAK1/STAT3 Pathway Induced by TGF-β in Human LX-2 Cells

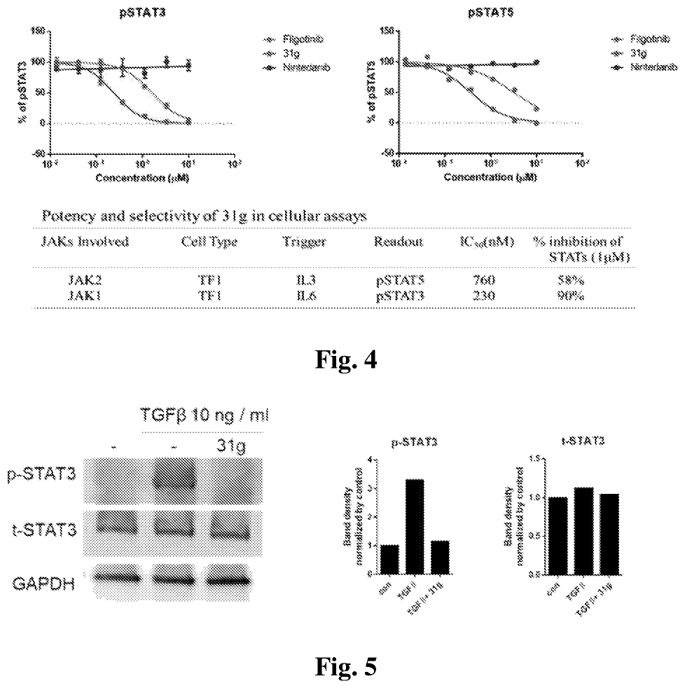

Immunobit cell-based immunoassays revealed that 31g inhibited JAK1/STAT3 signaling in HSCs in a dose-dependent manner. In contrast, nintedanib did not affect the JAK/STAT pathway (FIG. 4). In the same experiment, filgotinib, a well-known JAK1-selective inhibitor, also reduced pSTAT3 and pSTAT5, but with less effect than 31g.

These results indicate that both nintedanib and 31g ameliorate liver fibrosis (through reduction of fibrotic parameters and gene expression and migration) but they function through different pathways.

In conclusion, 31g could be a substance that is effective alone for liver fibrosis or a concomitant drug that exerts a synergistic effect on liver fibrosis due to the combination with other less toxic pathways.

### ACKNOWLEDGMENT

The present invention was made with the support of Korea Pharma Co., Ltd. and the National Research Foundation of Korea (Project Nos.: 2019M3E5D4065251; 2018R1A5A2025286).

1. A compound represented by Formula I: 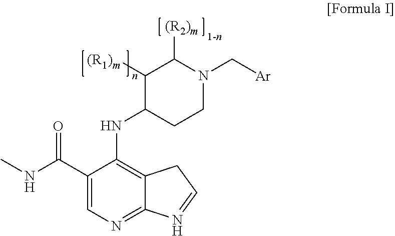
wherein
R₁ is C₁-C₃ alkyl or halogen,
R₂ is C₁-C₃ alkyl,
Ar is phenyl or heteroaryl unsubstituted or substituted with one or more substituents selected from the group consisting of C₁-C₃ alkyl, C₁-C₃ alkoxy, halogen, cyan, and —CF₃,
m is 1 or 2, and n is 0 or 1, or a pharmaceutically acceptable salt thereof.

2. The compound or pharmaceutically acceptable salt thereof according to claim 1, wherein the compound of Formula I is represented by Formula II:

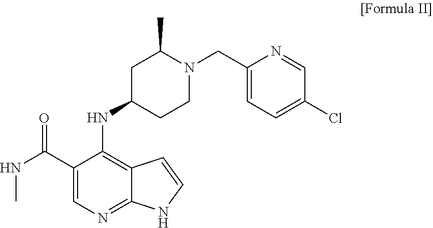 [SMILES: CNC(=O)c1cnc2[nH]ccc2c1N[C@@H]1CCN(Cc2ccc(Cl)cn2)[C@H](C)C1]

3. The compound or pharmaceutically acceptable salt thereof according to claim 1, wherein the compound of Formula I is represented by Formula III:

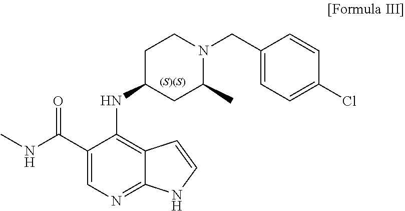

4. The compound or pharmaceutically acceptable salt thereof according to claim 1, wherein the compound of Formula I is represented by Formula IV:

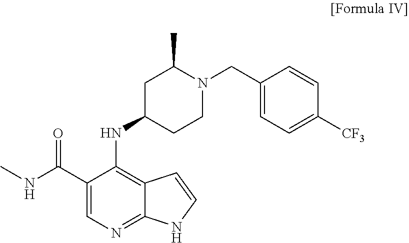 [SMILES: CNC(=O)c1cnc2[nH]ccc2c1N[C@@H]1CCN(Cc2ccc(C(F)(F)F)cc2)[C@H](C)C1]

5. The compound or pharmaceutically acceptable salt thereof according to claim 1, wherein the compound of Formula I is represented by Formula V: 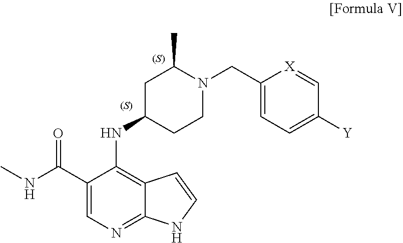
wherein X is CH or nitrogen and Y is C₁-C₃ alkyl, C₁-C₃ alkoxy, halogen, cyan or —CF₃.

6. A pharmaceutical composition for treating or preventing an autoimmune disease or cancer comprising the compound or pharmaceutically acceptable salt thereof according to any one of claims 1.

7. The pharmaceutical composition according to claim 6, wherein the autoimmune disease is atopic dermatitis, psoriasis, skin rash, contact dermatitis, multiple sclerosis, rheumatoid arthritis, psoriatic arthritis, juvenile arthritis, type I diabetes, lupus, inflammatory bowel disease, Crohn's disease, severe myasthenia gravis, immunoglobulin nephropathy, myocarditis or autoimmune thyroid disorders.

8. The pharmaceutical composition according to claim 6, wherein the cancer is pancreatic cancer, prostate cancer, lung cancer, head and neck cancer, breast cancer, colon cancer, ovarian cancer, gastric cancer, hepatic cancer, Castleman's disease, multiple myeloma, lymphoma, melanoma, neuroblastoma, glioblastoma, systemic mastocytosis or leukemia.

9. A pharmaceutical composition for treating or preventing liver fibrosis comprising the compound or pharmaceutically acceptable salt thereof according to any one of claims 1.
